(19) United States
(12) Patent Application Publication
(10) Pub. No.: US 2025/0281281 A1
(43) Pub. Date: Sep. 11, 2025

(54) PROSTHETIC VALVE DOCKING DEVICE

(71) Applicant: EDWARDS LIFESCIENCES CORPORATION, Irvine, CA (US)

(72) Inventors: Jocelyn Chau, Buena Park, CA (US); Tram Ngoc Nguyen, Santa Ana, CA (US)

(73) Assignee: EDWARDS LIFESCIENCES CORPORATION, Irvine, CA (US)

(21) Appl. No.: 19/213,702

(22) Filed: May 20, 2025

Related U.S. Application Data
(63) Continuation of application No. PCT/US2023/082578, filed on Dec. 5, 2023.
(60) Provisional application No. 63/387,911, filed on Dec. 16, 2022.

(51) Int. Cl. A61F 2/24

(52) U.S. Cl. CPC A61F 2/24; A61F 2220/0008; A61F 2230/0091

(57) ABSTRACT

Example docking devices for securing a prosthetic valve at a native valve are disclosed. In certain examples, a docking device can include a coil having a plurality of helical turns when deployed at the native valve, and a retention member covering at least a portion of the coil. The retention member can include a first axial segment. In certain examples, the first axial segment encloses only a partial circumference of the coil. In certain examples, the first axial segment includes a first circumferential portion and a second circumferential portion, and the second circumferential portion has a smoother outer surface than the first circumferential portion.

### CROSS-REFERENCE TO RELATED APPLICATIONS

This application is a continuation of PCT Patent Application No. PCT/US2023/082578, filed Dec. 5, 2023, which claims the benefit of U.S. Provisional Application No. 63/387,911, filed Dec. 16, 2022, each of which is incorporated by reference in its entirety herein.

### FIELD

The present disclosure concerns examples of a docking device configured to secure a prosthetic valve at a native heart valve, as well as methods of assembling such devices.

### BACKGROUND

Prosthetic valves can be used to treat cardiac valvular disorders. Native heart valves (for example, the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (for example, a native aortic valve). As such, the THV may not be suitable for implantation at another native valve (for example, a native mitral valve) and/or in a patient with a larger native valve. Additionally, or alternatively, the native tissue at the implantation site may not provide sufficient structure for the THV to be secured in place relative to the native tissue. Accordingly, improvements to THVs and the associated transcatheter delivery apparatus are desirable.

### SUMMARY

The present disclosure relates to methods and devices for treating valvular regurgitation and/or other valve issues. Specifically, the present disclosure is directed to a docking device configured to receive a prosthetic valve and the methods of assembling the docking device and implanting the docking device.

A docking device for securing a prosthetic valve at a native valve can include a coil comprising a plurality of helical turns when deployed at the native valve. In addition to these features, a docking device can further comprise one or more of the components disclosed herein.

In certain examples, a docking device can include a retention member covering at least a portion of the coil.

In certain examples, the retention member includes a first axial segment which encloses only a partial circumference of the coil.

In certain examples, the retention member includes a first axial segment which includes a first circumferential portion and a second circumferential portion. The second circumferential portion has a smoother outer surface than the first circumferential portion.

Certain aspects of the disclosure concern an implant assembly including a radially expandable and compressible prosthetic valve and the docking device described above.

A method for assembling a docking device configured to receive a prosthetic valve can include preparing a retention member having a first axial segment and a second axial segment. The method can further include attaching the retention member to a coil of the docking device so that the first axial segment encloses only a partial circumference of the coil and the second axial segment encloses a full circumference of the coil. The coil is movable from a substantially straight configuration to a helical configuration.

A method for assembling a docking device configured to receive a prosthetic valve can include preparing a retention member having a tubular configuration. The retention member has a first axial segment and a second axial segment. The method can further include attaching the retention member to a coil of the docking device so that the retention member encloses a full circumference of the coil. The coil is movable from a substantially straight configuration to a helical configuration. The first axial segment has a first circumferential portion and a second circumferential portion. The second circumferential portion has a smoother outer surface than the first circumferential portion.

Certain aspects of the disclosure concern a method for implanting a prosthetic valve. The method can include deploying the docking device described above at a native valve, and deploying the prosthetic valve within the docking device.

The above method can be performed on a living animal or on a simulation, such as on a cadaver, cadaver heart, anthropomorphic ghost, simulator (for example, with body parts, heart, tissue, etc. being simulated).

In some examples, a docking device comprises one or more of the components recited in Examples 1-26 described in the section “Additional Examples of the Disclosed Technology” below.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a first stage in an exemplary mitral valve replacement procedure where a guide catheter and a guidewire are inserted into a vasculature of a patient and navigated through the vasculature and into a heart of the patient, towards a native mitral valve of the heart.

FIG. 2A schematically illustrates a second stage in the exemplary mitral valve replacement procedure where a docking device delivery apparatus extending through the guide catheter is used to deploy a docking device at the native mitral valve.

FIG. 2B schematically illustrates a third stage in the exemplary mitral valve replacement procedure where the docking device of FIG. 2A is fully implanted at the native mitral valve of the patient and the docking device delivery apparatus has been removed from the patient.

FIG. 3A schematically illustrates a fourth stage in the exemplary mitral valve replacement procedure where a prosthetic heart valve delivery apparatus extending through the guide catheter is deploy a prosthetic heart valve within the implanted docking device at the native mitral valve.

FIG. 3B schematically illustrates a fifth stage in the exemplary mitral valve replacement procedure where the prosthetic heart valve is fully implanted within the docking device at the native mitral valve and the prosthetic heart valve delivery apparatus has been removed from the patient.

FIG. 4 schematically illustrates a sixth stage in the exemplary mitral valve replacement procedure where the guide catheter and the guidewire have been removed from the patient.

FIG. 5A is a side perspective view of a docking device in a helical configuration, according to one example.

FIG. 5B is a top view of the docking device depicted in FIG. 5A.

FIG. 5C is a cross-sectional view of the docking device taken along line 5C-5C depicted in FIG. 5B, according to one example.

FIG. 5D is a cross-sectional view of the docking device taken along the same line as in FIG. 5C, except in FIG. 5D, the docking device is in a substantially straight delivery configuration.

FIG. 6A is a perspective view a prosthetic valve, according to one example.

FIG. 6B is a perspective view of the prosthetic valve of FIG. 6A with an outer cover, according to one example.

FIG. 7A is a perspective view of an exemplary prosthetic implant assembly comprising the docking device depicted in FIG. 5A and the prosthetic valve of FIG. 6B retained within the docking device.

FIG. 7B is a side elevation view of the prosthetic implant assembly of FIG. 7A

FIG. 8 depicts a section of an example docking device in a substantially straight delivery configuration (the guard member is removed).

FIG. 8A depicts a cross-section view of the docking device of FIG. 8 taken along the line 8A-8A, according to one example.

FIG. 8B depicts a cross-section view of the docking device of FIG. 8 taken along the line 8B-8B, according to one example.

FIG. 9 depicts a section of another example docking device in a substantially straight delivery configuration (the guard member is removed).

FIG. 9A depicts a cross-section view of the docking device of FIG. 9 taken along the line 9A-9A, according to one example.

FIG. 9B depicts a cross-section view of the docking device of FIG. 9 taken along the line 9B-9B, according to one example.

FIG. 10 is a side view of an example docking device in a substantially straight delivery configuration, where a retention member of the docking device has a circumferential gap.

FIG. 11 is a side view of an example docking device in a helical deployed configuration, where a retention member of the docking device has a smooth circumferential portion facing radially outwardly and a textured circumferential portion facing radially inwardly.

FIG. 12A depicts a holder device, according to one example.

FIG. 12B depicts a retention member retained in the holder device.

FIG. 12C depicts applying heat to smooth out an exposed portion of the retention member of FIG. 12B, according to one example.

FIG. 12D depicts the retention member of FIG. 12B after the heat treatment.

### DETAILED DESCRIPTION

### General Considerations

It should be understood that the disclosed examples can be adapted to deliver and implant prosthetic devices in any of the native annuluses of the heart (for example, the pulmonary, mitral, and tricuspid annuluses), and can be used with any of various delivery approaches (for example, retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (for example, mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

Directions and other relative references (for example, inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Exemplary Transcatheter Heart Valve Replacement Procedure

Described herein are various systems, apparatuses, methods, or the like, that can be used in or with delivery apparatuses to deliver a prosthetic implant (for example, a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (for example, a flush fluid, such as heparinized saline or the like) can be provided to a pusher shaft lumen defined within an interior of the pusher shaft, a delivery shaft lumen defined between the sleeve shaft and the outer shaft of the delivery apparatus, and a sleeve shaft lumen defined between the pusher shaft and the sleeve shaft. By providing a consistent flow of fluid through these lumens of the delivery apparatus, stagnation of blood within the delivery apparatus can be reduced or avoided, thereby reducing a risk of thrombus formation.

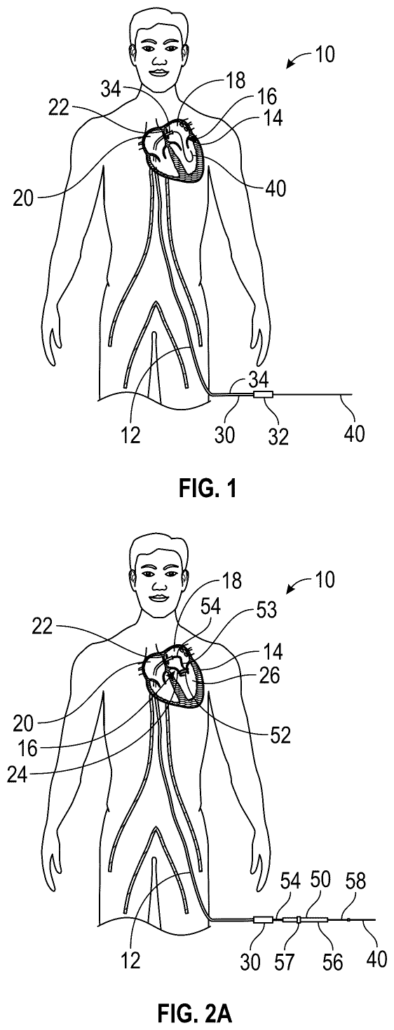

An exemplary transcatheter heart valve replacement procedure which utilizes a first delivery apparatus to deliver a docking device to a native valve annulus and then a second delivery apparatus to deliver a prosthetic heart valve (for example, THV) inside the docking device is depicted in the schematic illustrations of FIGS. 1-4.

As introduced above, defective native heart valves may be replaced with THVs. However, in certain instances, such THVs may not be able to sufficiently secure themselves to the native tissue (for example, to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

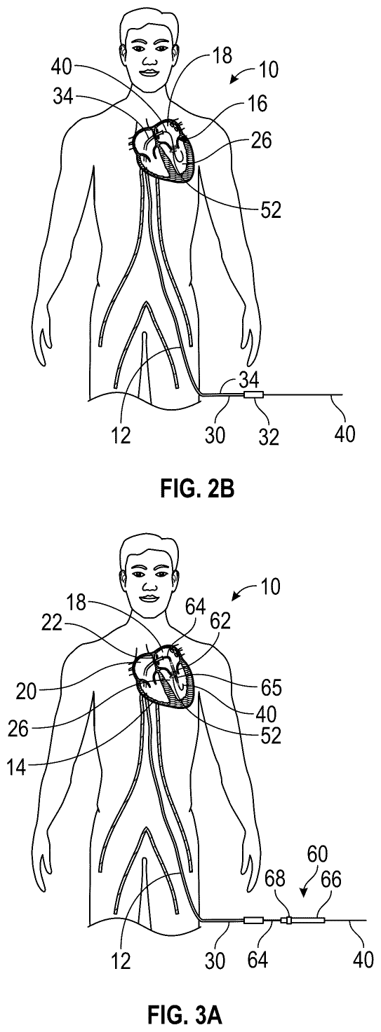

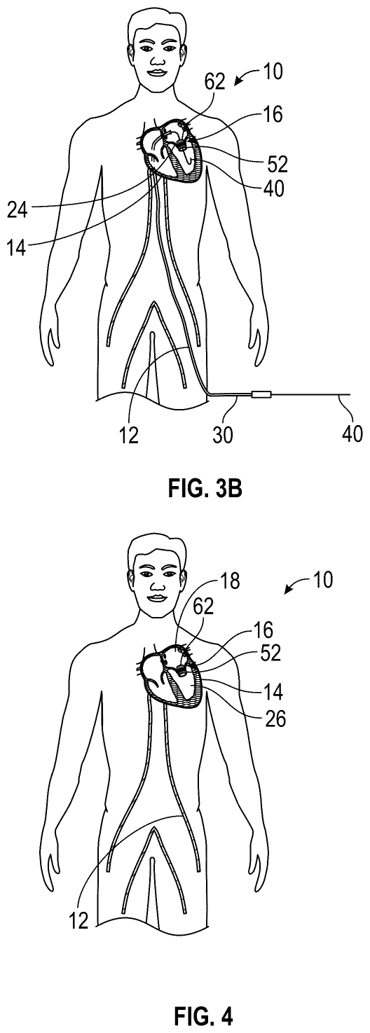

FIGS. 1-4 depict an exemplary transcatheter heart valve replacement procedure (for example, a mitral valve replacement procedure) which utilizes a docking device 52 and a prosthetic heart valve 62, according to one example. During the procedure, a user can create a pathway to a patient's native heart valve using a guide catheter 30 (FIG. 1). The user can deliver and implant the docking device 52 at the patient's native heart valve using a docking device delivery apparatus 50 (FIG. 2A) and then removes the docking device delivery apparatus 50 from the patient 10 after implanting the docking device 52 (FIG. 2B). The user can then implant the prosthetic heart valve 62 within the implanted docking device 52 using a prosthetic valve delivery apparatus 60 (FIG. 3A). Thereafter, the user can remove the prosthetic valve delivery apparatus 60 from the patient 10 (FIG. 3B), as well as the guide catheter 30 (FIG. 4).

FIG. 1 depicts a first stage in a mitral valve replacement procedure, according to one example. As shown, the guide catheter 30 and a guidewire 40 can be inserted into a vasculature 12 of a patient 10 and navigated through the vasculature 12, into a heart 14 of the patient 10, and toward the native mitral valve 16. Together, the guide catheter 30 and the guidewire 40 can provide a path for the docking device delivery apparatus 50 and the prosthetic valve delivery apparatus 60 to be navigated through and along, to the implantation site (for example, the native mitral valve 16 or native mitral valve annulus).

Initially, the user may first make an incision in the patient's body to access the vasculature 12. For example, as illustrated in FIG. 1, the user may make an incision in the patient's groin to access a femoral vein. Thus, in such examples, the vasculature 12 may include a femoral vein.

After making the incision to access the vasculature 12, the user may insert the guide catheter 30, the guidewire 40, and/or additional devices (such as an introducer device or transseptal puncture device) through the incision and into the vasculature 12. The guide catheter 30 (which can also be referred to as an “introducer device,” “introducer,” or “guide sheath”) can be configured to facilitate the percutaneous introduction of various implant delivery devices (for example, the docking device delivery apparatus 50 and the prosthetic valve delivery apparatus 60) into and through the vasculature 12 and may extend through the vasculature 12 and into the heart 14 but may stop short of the native mitral valve 16. The guide catheter 30 can comprise a handle 32 and a shaft 34 extending distally from the handle 32. The shaft 34 can extend through the vasculature 12 and into the heart 14 while the handle 32 can remain outside the body of the patient 10 and can be operated by the user in order to manipulate the shaft 34 (FIG. 1).

The guidewire 40 can be configured to guide the delivery apparatuses (for example, the guide catheter 30, the docking device delivery apparatus 50, the prosthetic valve delivery apparatus 60, additional catheters, or the like) and their associated devices (for example, docking device, prosthetic heart valve, and the like) to the implantation site within the heart 14, and thus may extend all the way through the vasculature 12 and into a left atrium 18 of the heart 14 (and in some examples, through the native mitral valve 16 and into a left ventricle of the heart 14) (FIG. 1).

In some instances, a transseptal puncture device or catheter can be used to initially access the left atrium 18, prior to inserting the guidewire 40 and the guide catheter 30. For example, after making the incision to access the vasculature 12, the user may insert a transseptal puncture device through the incision and into the vasculature 12. The user may guide the transseptal puncture device through the vasculature 12 and into the heart 14 (for example, through the femoral vein and into the right atrium 20). The user can then make a small incision in an atrial septum 22 of the heart 14 to allow access to the left atrium 18 from the right atrium 20. The user can then insert and advance the guidewire 40 through the transseptal puncture device within the vasculature 12 and through the incision in the atrial septum 22 into the left atrium 18. Once the guidewire 40 is positioned within the left atrium 18 and/or the left ventricle 26, the transseptal puncture device can be removed from the patient 10. The user can then insert the guide catheter 30 into the vasculature 12 and advance the guide catheter 30 into the left atrium 18 over the guidewire 40 (FIG. 1).

In some instances, an introducer device can be inserted through a lumen of the guide catheter 30 prior to inserting the guide catheter 30 into the vasculature 12. In some instances, the introducer device can include a tapered end that extends out a distal tip of the guide catheter 30 and that is configured to guide the guide catheter 30 into the left atrium 18 over the guidewire 40. Additionally, in some instances the introducer device can include a proximal end portion that extends out a proximal end of the guide catheter 30. Once the guide catheter 30 reaches the left atrium 18, the user can remove the introducer device from inside the guide catheter 30 and the patient 10. Thus, only the guide catheter 30 and the guidewire 40 remain inside the patient 10. The guide catheter 30 is then in position to receive an implant delivery apparatus and help guide it to the left atrium 18, as described further below.

FIG. 2A depicts a second stage in the exemplary mitral valve replacement procedure where a docking device 52 can be implanted at the native mitral valve 16 of the heart 14 of the patient 10 using a docking device delivery apparatus 50 (which may also be referred to as an “implant catheter,” or a “docking device delivery device,” or simply “delivery apparatus”).

In general, the docking device delivery apparatus 50 can include a delivery shaft 54 (which may also be referred to as an “outer shaft”), a handle 56, and a pusher assembly 58. The delivery shaft 54 can be configured to be advanced through the patient's vasculature 12 and to the implantation site (for example, native mitral valve 16) by the user, and may be configured to retain the docking device 52 in a distal end portion 53 of the delivery shaft 54. In some examples, the distal end portion 53 of the delivery shaft 54 can retain the docking device 52 therein in a substantially straightened delivery configuration.

The handle 56 of the docking device delivery apparatus 50 can be configured to be gripped and/or otherwise held by the user to advance the delivery shaft 54 through the patient's vasculature 12. Specifically, the handle 56 can be coupled to a proximal end of the delivery shaft 54 and can be configured to remain accessible to the user (for example, outside the body of the patient 10) during the docking device implantation procedure. In this way, the user can advance the delivery shaft 54 through the patient's vasculature 12 by exerting a force on (for example, pushing) the handle 56. In some examples, the delivery shaft 54 can be configured to carry the pusher assembly 58 and/or the docking device 52 with it as it advances through the patient's vasculature 12. In this way, the docking device 52 and/or the pusher assembly 58 can advance through the patient's vasculature 12 in lockstep with the delivery shaft 54 as the user grips the handle 56 and pushes the delivery shaft 54 deeper into the patient's vasculature 12.

In some examples, the handle 56 can comprise one or more articulation members 57 that are configured to aid in navigating the delivery shaft 54 through the vasculature 12. For example, the one or more articulation members 57 can comprise one or more of knobs, buttons, wheels, and/or other types of physically adjustable control members that are configured to be adjusted by the user to flex, bend, twist, turn, and/or otherwise articulate a distal end portion 53 of the delivery shaft 54 to aid in navigating the delivery shaft 54 through the vasculature 12 and/or within the heart 14.

The pusher assembly 58 can be configured to deploy and/or implant the docking device 52 at the implantation site (for example, the native mitral valve 16). For example, the pusher assembly 58 can be configured to be adjusted by the user to push the docking device 52 out of the distal end portion 53 of the delivery shaft 54. A pusher shaft of the pusher assembly 58 can extend through the delivery shaft 54 and can be disposed adjacent to the docking device 52 within the delivery shaft 54. In some examples, the docking device 52 can be releasably coupled to the pusher shaft of the pusher assembly 58 via a connection mechanism of the docking device delivery apparatus 50 such that the docking device 52 can be released after being deployed at the native mitral valve 16. Because the docking device 52 is retained by, held, and/or otherwise coupled to the pusher assembly 58, the docking device 52 can advance in lockstep with the pusher assembly 58 through and/or out of the delivery shaft 54.

In addition to the pusher shaft, in certain instances, the pusher assembly 58 can also include a sleeve shaft. The pusher shaft can be configured to advance the docking device 52 through the delivery shaft 54 and out of the distal end portion 53 of the delivery shaft 54, while the sleeve shaft, when included, can have a distal dock sleeve configured to cover the docking device 52 within the delivery shaft 54 and while pushing the docking device 52 out of the delivery shaft 54 and positioning the docking device 52 at the implantation site. In some examples, the pusher shaft can be covered, at least in part, by the sleeve shaft. In certain examples, the sleeve shaft can include telescopic shaft members, as described further below.

In some examples, the pusher assembly 58 can comprise a pusher handle that is coupled to the pusher shaft and that is configured to be gripped and pushed by the user to translate the pusher shaft axially relative to the delivery shaft 54 (for example, to push the pusher shaft into and/or out of the distal end portion 53 of the delivery shaft 54). The dock sleeve can be configured to be retracted and/or withdrawn from the docking device 52, after positioning the docking device 52 at the target implantation site. For example, the pusher assembly 58 can include a sleeve handle that is coupled to the sleeve shaft and is configured to be pulled by a user to retract (for example, axially move) the sleeve shaft relative to the pusher shaft, thereby retracting the dock sleeve.

The pusher assembly 58 can be removably coupled to the docking device 52, and as such can be configured to release, detach, decouple, and/or otherwise disconnect from the docking device 52 once the docking device 52 has been deployed at the target implantation site. As just one example, the pusher assembly 58 may be removably coupled to the docking device 52 via a thread, string, yarn, suture, or other suitable material that is tied or sutured to the docking device 52.

In some examples, the pusher assembly 58 can include a suture lock assembly (also referred to as a “suture lock”) that is configured to receive and/or hold the thread or other suitable material that is coupled to the docking device 52 via a suture. The thread or other suitable material that forms the suture can extend from the docking device 52, through the pusher assembly 58, to the suture lock assembly. The suture lock assembly can also be configured to cut the suture to release, detach, decouple, and/or otherwise disconnect the docking device 52 from the pusher assembly 58. For example, the suture lock assembly can comprise a cutting mechanism that is configured to be adjusted by the user to cut the suture.

Referring again to FIG. 2A, after the guide catheter 30 is positioned within the left atrium 18, the user may insert the docking device delivery apparatus 50 (for example, the delivery shaft 54) into the patient 10 by advancing the delivery shaft 54 of the docking device delivery apparatus 50 through the guide catheter 30 and over the guidewire 40. In some examples, the guidewire 40 can be at least partially retracted away from the left atrium 18 and into the guide catheter 30. The user may then continue to advance the delivery shaft 54 of the docking device delivery apparatus 50 through the vasculature 12 along the guidewire 40 until the delivery shaft 54 reaches the left atrium 18, as illustrated in FIG. 2A. Specifically, the user may advance the delivery shaft 54 of the docking device delivery apparatus 50 by gripping and exerting a force on (for example, pushing) the handle 56 of the docking device delivery apparatus 50 toward the patient 10. While advancing the delivery shaft 54 through the vasculature 12 and the heart 14, the user may adjust the one or more articulation members 57 of the handle 56 to navigate the various turns, corners, constrictions, and/or other obstacles in the vasculature 12 and the heart 14.

Once the delivery shaft 54 reaches the left atrium 18 and extends out of a distal end of the guide catheter 30, the user can position the distal end portion 53 of the delivery shaft 54 at and/or near the posteromedial commissure of the native mitral valve 16 using the handle 56 (for example, the articulation members 57). The user may then push the docking device 52 out of the distal end portion 53 of the delivery shaft 54 with the shaft of the pusher assembly 58 to deploy and/or implant the docking device 52 within the annulus of the native mitral valve 16.

In some examples, the docking device 52 may be constructed from, formed of, and/or comprise a shape memory material, and as such, may return to its original, pre-formed shape when it exits the delivery shaft 54 and is no longer constrained by the delivery shaft 54. As one example, the docking device 52 may originally be formed as a coil, and thus may wrap around leaflets 24 of the native mitral valve 16 as it exits the delivery shaft 54 and returns to its original coiled configuration.

After pushing a ventricular portion of the docking device 52 (for example, the portion of the docking device 52 shown in FIG. 2A that is configured to be positioned within a left ventricle 26 and/or on the ventricular side of the native mitral valve 16), the user may then deploy the remaining portion of the docking device 52 (for example, an atrial portion of the docking device 52) from the delivery shaft 54 within the left atrium 18 by retracting the delivery shaft 54 away from the posteromedial commissure of the native mitral valve 16. For example, the user can maintain the position of the pusher assembly 58 (for example, by exerting a holding and/or pushing force on the pusher shaft) while retracting the delivery shaft 54 proximally so that the delivery shaft 54 withdraws and/or otherwise retracts relative to the docking device 52 and the pusher assembly 58. In this way, the pusher assembly 58 can hold the docking device 52 in place while the user retracts the delivery shaft 54, thereby releasing the docking device 52 from the delivery shaft 54. In some examples, the user can also remove the dock sleeve from the docking device 52, for example, by retracting the sleeve shaft.

After deploying and implanting the docking device 52 at the native mitral valve 16, the user may disconnect the docking device delivery apparatus 50 from the docking device 52. Once the docking device 52 can be disconnected from the docking device delivery apparatus 50 (for example, by cutting the suture tied to the docking device 52), the user may retract the docking device delivery apparatus 50 out of the vasculature 12 and away from the patient 10 so that the user can deliver and implant a prosthetic heart valve 62 within the implanted docking device 52 at the native mitral valve 16.

FIG. 2B depicts a third stage in the mitral valve replacement procedure, where the docking device 52 has been fully deployed and implanted at the native mitral valve 16 and the docking device delivery apparatus 50 (including the delivery shaft 54) has been removed from the patient 10 such that only the guidewire 40 and the guide catheter 30 remain inside the patient 10. In some examples, after removing the docking device delivery apparatus, the guidewire 40 can be advanced out of the guide catheter 30, through the implanted docking device 52 at the native mitral valve 16, and into the left ventricle 26 (FIG. 2A). As such, the guidewire 40 can help to guide the prosthetic valve delivery apparatus 60 through the annulus of the native mitral valve 16 and at least partially into the left ventricle 26.

As illustrated in FIG. 2B, the docking device 52 can comprise a plurality of helical turns that wrap around the leaflets 24 of the native mitral valve 16 (within the left ventricle 26). The implanted docking device 52 can have a more cylindrical shape than the annulus of the native mitral valve 16, thereby providing a geometry that more closely matches the shape or profile of the prosthetic heart valve to be implanted. As a result, the docking device 52 can provide a tighter fit, and thus a better seal, between the prosthetic heart valve and the native mitral valve 16, as described further below.

FIG. 3A depicts a fourth stage in the mitral valve replacement procedure where the user is delivering and/or implanting a prosthetic heart valve 62 within the docking device 52 using a prosthetic valve delivery apparatus 60.

As shown in FIG. 3A, the prosthetic valve delivery apparatus 60 can comprise a delivery shaft 64 and a handle 66. The delivery shaft 64 can extend distally from the handle 66. The delivery shaft 64 can be configured to extend into the patient's vasculature 12 to deliver, implant, expand, and/or otherwise deploy the prosthetic heart valve 62 within the docking device 52 at the native mitral valve 16. The handle 66 can be configured to be gripped and/or otherwise held by the user to advance the delivery shaft 64 through the patient's vasculature 12.

In some examples, the handle 66 can comprise one or more articulation members 68 that are configured to aid in navigating the delivery shaft 64 through the vasculature 12 and the heart 14. Specifically, the articulation members 68 can comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the vasculature 12 and into the left atrium 18 and left ventricle 26 of the heart 14.

In some examples, the prosthetic valve delivery apparatus 60 can include an expansion mechanism 65 that is configured to radially expand and deploy the prosthetic heart valve 62 at the implantation site. In some instances, as shown in FIG. 3A, the expansion mechanism 65 can comprise an inflatable balloon that is configured to be inflated to radially expand the prosthetic heart valve 62 within the docking device 52. The inflatable balloon can be coupled to the distal end portion of the delivery shaft 64.

In other examples, the prosthetic heart valve 62 can be self-expanding and can be configured to radially expand on its own upon removable of a sheath or capsule covering the radially compressed prosthetic heart valve 62 on the distal end portion of the delivery shaft 64. In still other examples, the prosthetic heart valve 62 can be mechanically expandable and the prosthetic valve delivery apparatus 60 can include one or more mechanical actuators (for example, the expansion mechanism) configured to radially expand the prosthetic heart valve 62.

As shown in FIG. 3A, the prosthetic heart valve 62 can be mounted around the expansion mechanism 65 (for example, the inflatable balloon) on the distal end portion of the delivery shaft 64, in a radially compressed configuration.

To navigate the distal end portion of the delivery shaft 64 to the implantation site, the user can insert the prosthetic valve delivery apparatus 60 (for example, the delivery shaft 64) into the patient 10 through the guide catheter 30 and over the guidewire 40. The user can continue to advance the prosthetic valve delivery apparatus 60 along the guidewire 40 (for example, through the vasculature 12) until the distal end portion of the delivery shaft 64 reaches the native mitral valve 16, as illustrated in FIG. 3A. More specifically, the user can advance the delivery shaft 64 of the prosthetic valve delivery apparatus 60 by gripping and exerting a force on (for example, pushing) the handle 66. While advancing the delivery shaft 64 through the vasculature 12 and the heart 14, the user can adjust the one or more articulation members 68 of the handle 66 to navigate the various turns, corners, constrictions, and/or other obstacles in the vasculature 12 and heart 14.

The user can advance the delivery shaft 64 along the guidewire 40 until the radially compressed prosthetic heart valve 62 mounted around the distal end portion of the delivery shaft 64 is positioned within the docking device 52 and the native mitral valve 16. In some examples, as shown in FIG. 3A, a distal end of the delivery shaft 64 and a least a portion of the radially compressed prosthetic heart valve 62 can be positioned within the left ventricle 26.

Once the radially compressed prosthetic heart valve 62 is appropriately positioned within the docking device 52 (FIG. 3A), the user can manipulate one or more actuation mechanisms of the handle 66 of the prosthetic valve delivery apparatus 60 to actuate the expansion mechanism 65 (for example, inflate the inflatable balloon), thereby radially expanding the prosthetic heart valve 62 within the docking device 52. In some examples, the user can lock the prosthetic heart valve 62 in its fully expanded position (for example, with a locking mechanism) to prevent the prosthetic heart valve 62 from collapsing.

FIG. 3B shows a fifth stage in the mitral valve replacement procedure where the prosthetic heart valve 62 in its radially expanded configuration and implanted within the docking device 52 in the native mitral valve 16. As shown in FIG. 3B, the prosthetic heart valve 62 can be received and retained within the docking device 52.

As also shown in FIG. 3B, after the prosthetic heart valve 62 has been fully deployed and implanted within the docking device 52 at the native mitral valve 16, the prosthetic valve delivery apparatus 60 (including the delivery shaft 64) can be removed from the patient 10 such that only the guidewire 40 and the guide catheter 30 remain inside the patient 10.

FIG. 4 depicts a sixth stage in the mitral valve replacement procedure, where the guidewire 40 and the guide catheter 30 have been removed from the patient 10. The docking device 52 can be configured to provide a seal between the prosthetic heart valve 62 and the leaflets 24 of the native mitral valve 16 to reduce paravalvular leakage around the prosthetic heart valve 62. Specifically, the docking device 52 can initially constrict the leaflets 24 of the native mitral valve 16. The prosthetic heart valve 62 can then push the leaflets 24 against the docking device 52 as it radially expands within the docking device 52. Thus, the docking device 52 and the prosthetic heart valve 62 can be configured to sandwich the leaflets 24 of the native mitral valve 16 when the prosthetic heart valve 62 is expanded within the docking device 52. In this way, the docking device 52 can provide a seal between the leaflets 24 of the native mitral valve 16 and the prosthetic heart valve 62 to reduce paravalvular leakage around the prosthetic heart valve 62.

In some examples, one or more of the docking device delivery apparatus 50, the prosthetic valve delivery apparatus 60, and/or the guide catheter 30 can comprise one or more fluid ports that are configured to supply flushing fluid to the lumens thereof to prevent and/or reduce the likelihood of blood clot (for example, thrombus) formation. Example fluid ports that can be used to inject flushing fluid into a docking device delivery apparatus are described further below.

Although FIGS. 1-4 specifically depict a mitral valve replacement procedure, it should be appreciated that the same and/or similar procedure may be utilized to replace other heart valves (for example, tricuspid, pulmonary, and/or aortic valves). Further, the same and/or similar delivery apparatuses (for example, docking device delivery apparatus 50, prosthetic valve delivery apparatus 60, guide catheter 30, and/or guidewire 40), docking devices (for example, docking device 52), replacement heart valves (for example, prosthetic heart valve 62), and/or components thereof may be utilized for replacing these other heart valves.

For example, when replacing a native tricuspid valve, the user may also access the right atrium 20 via a femoral vein but may not need to cross the atrial septum 22 into the left atrium 18. Instead, the user may leave the guidewire 40 in the right atrium 20 and perform the same and/or similar docking device implantation process at the tricuspid valve. Specifically, the user may push the docking device 52 out of the delivery shaft 54 around the ventricular side of the tricuspid valve leaflets, release the remaining portion of the docking device 52 from the delivery shaft 54 within the right atrium 20, and then remove the delivery shaft 54 of the docking device delivery apparatus 50 from the patient 10. The user may then advance the guidewire 40 through the tricuspid valve into the right ventricle and perform the same and/or similar prosthetic heart valve implantation process at the tricuspid valve, within the docking device 52. Specifically, the user may advance the delivery shaft 64 of the prosthetic valve delivery apparatus 60 through the patient's vasculature along the guidewire 40 until the prosthetic heart valve 62 is positioned or disposed within the docking device 52 and the tricuspid valve. The user may then expand the prosthetic heart valve 62 within the docking device 52 before removing the prosthetic valve delivery apparatus 60 from the patient 10. In another example, the user may perform the same and/or similar process to replace the aortic valve but may access the aortic valve from the outflow side of the aortic valve via a femoral artery.

Further, although FIGS. 1-4 depict a mitral valve replacement procedure that accesses the native mitral valve 16 from the left atrium 18 via the right atrium 20 and femoral vein, it should be appreciated that the native mitral valve 16 may alternatively be accessed from the left ventricle 26. For example, the user may access the native mitral valve 16 from the left ventricle 26 via the aortic valve by advancing one or more delivery apparatuses through an artery to the aortic valve, and then through the aortic valve into the left ventricle 26.

Additional examples of the docking device delivery apparatus, including its variants, and methods of implanting a docking device and implanting a prosthetic valve within the docking device are described in PCT Patent Application Publication Nos. WO 2020/247907 and WO 2022/087336, and U.S. Patent Publication Nos. US2018/0318079, US2018/0263764, and US2018/0177594, which are all incorporated by reference herein in their entireties.

### Exemplary Docking Devices

Docking devices according to examples of the disclosure can, for example, provide a stable anchoring site, landing zone, or implantation zone at the implant site in which prosthetic valves can be expanded or otherwise implanted. Many of the disclosed docking devices comprise a circular or cylindrically-shaped portion, which can (for example) allow a prosthetic heart valve comprising a circular or cylindrically-shaped valve frame to be expanded or otherwise implanted into native locations with naturally circular cross-sectional profiles and/or in native locations with naturally with non-circular cross sections. In addition to providing an anchoring site for the prosthetic valve, the docking devices can be sized and shaped to cinch or draw the native valve (for example, mitral, tricuspid, etc.) anatomy radially inwards. In this manner, one of the main causes of valve regurgitation (for example, functional mitral regurgitation), specifically enlargement of the heart (for example, enlargement of the left ventricle, etc.) and/or valve annulus, and consequent stretching out of the native valve (for example, mitral, etc.) annulus, can be at least partially offset or counteracted. Some examples of the docking devices further include features which, for example, are shaped and/or modified to better hold a position or shape of the docking device during and/or after expansion of a prosthetic valve therein. By providing such docking devices, replacement valves can be more securely implanted and held at various valve annuluses, including at the mitral valve annulus which does not have a naturally circular cross-section.

In some instances, a docking device can comprise a paravalvular leakage (PVL) guard (also referred to herein as “a guard member”). The PVL guard can, for example, help reduce regurgitation and/or promote tissue ingrowth between the native tissue and the docking device, thereby reducing or preventing migration of the docking device (and the prosthetic valve received within the docking device) relative to the native tissue.

The PVL guard can, in some examples, be movable between a delivery configuration and a deployed configuration. When the PVL guard is in the delivery configuration, an outer edge of the PVL guard can extend along and adjacent the coil. When the PVL guard is in the deployed configuration, the outer edge of the PVL guard can form a helical shape rotating about a central longitudinal axis of the coil and at least a segment of the outer edge of PVL guard can extend radially away from the coil.

In certain examples, the PVL guard can cover or surround a portion of a coil of the docking device. As described more fully below, such PVL guard can move from a radially compressed (and axially elongated) state to a radially expanded (and axially foreshortened) state, and a proximal end portion of the PVL guard can be axially movable relative to the coil.

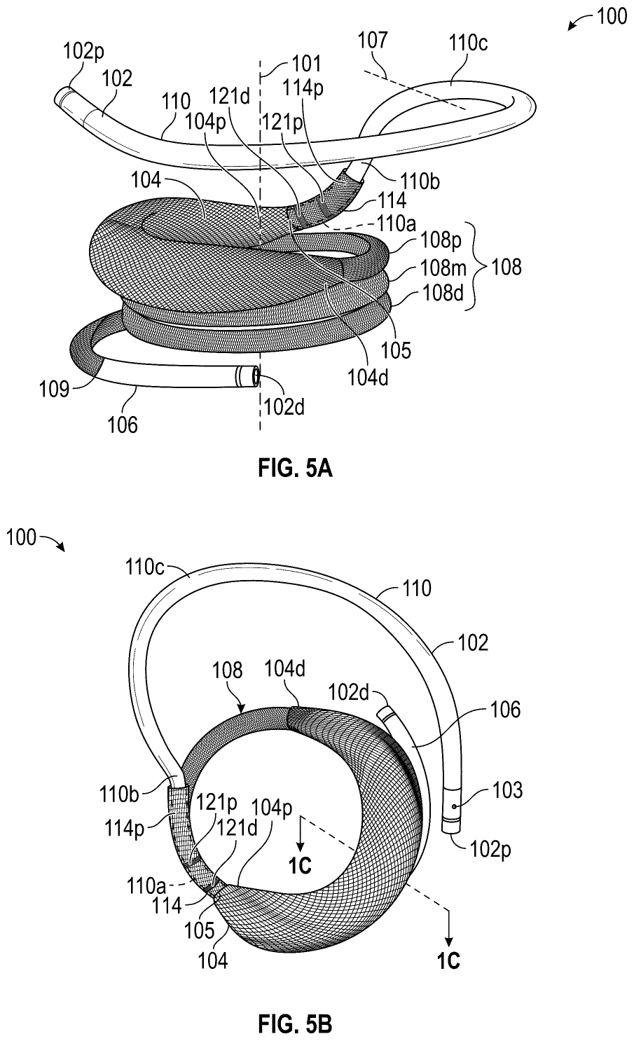

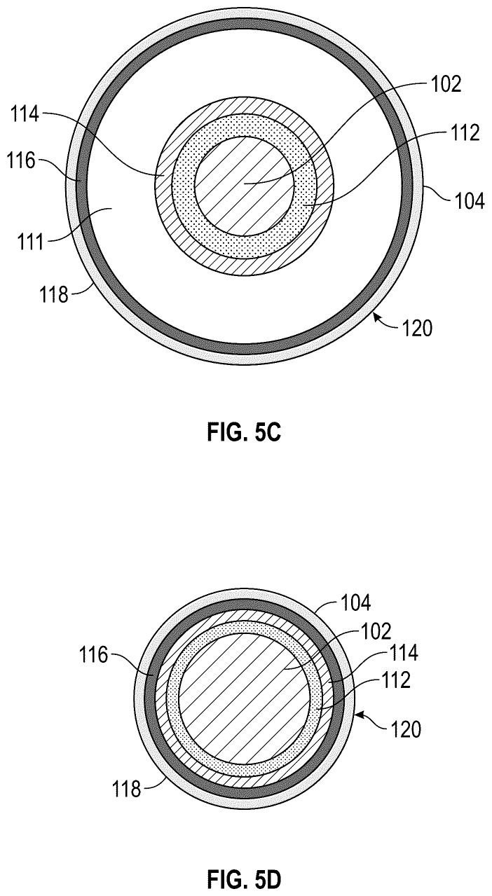

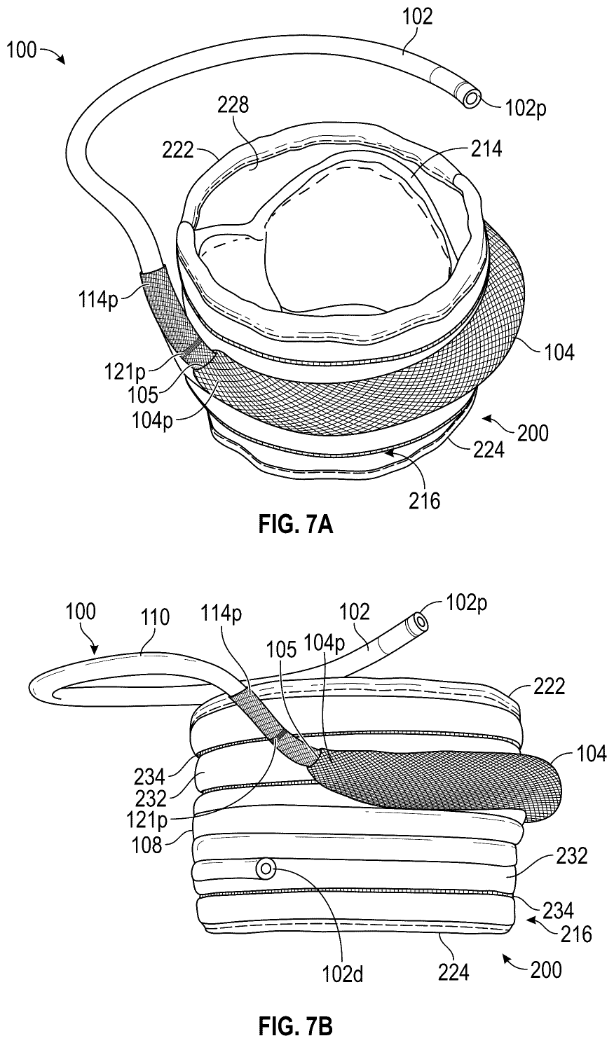

FIGS. 5A-5D show a docking device 100, according to one example. The docking device 100 can, for example, be implanted within a native valve annulus. As depicted in FIGS. 7A-7B, the docking device can be configured to receive and secure a prosthetic valve within the docking device, thereby securing the prosthetic valve at the native valve annulus.

Referring to FIGS. 5A-5D, the docking device 100 can comprise a coil 102 and a guard member 104 covering at least a portion of the coil 102. In certain examples, the coil 102 can include a shape memory material (for example, nickel titanium alloy or “Nitinol”) such that the docking device 100 (and the coil 102) can move from a substantially straight configuration (also referred to as “delivery configuration”) when disposed within a delivery sheath of a delivery apparatus to a helical configuration (also referred to as “deployed configuration,” as shown in FIGS. 5A-5B) after being removed from the delivery sheath.

In certain examples, when the guard member 104 is in the deployed configuration, the guard member 104 can extend circumferentially relative to a central longitudinal axis 101 of the docking device 100 from 180 degrees to 400 degrees, or from 210 degrees to 330 degrees, or from 250 degrees to 290 degrees, or from 260 degrees to 280 degrees. In one particularly example, when the guard member 104 is in the deployed configuration, the guard member 104 can extend circumferentially 270 degrees relative to the central longitudinal axis 101. In other words, the guard member 104 can extend circumferentially from about one half of a revolution (for example, 180 degrees) around the central longitudinal axis 101 in some examples to more than a full revolution (for example, 400 degrees) around the central longitudinal axis 101 in other examples, including various ranges in between. As used herein, a range (for example, 180-400 degrees, from 180 degrees to 400 degrees, and between 180 degrees and 400 degrees) includes the endpoints of the range (for example, 180 degrees and 400 degrees).

In some examples, the docking device 100 can also include a retention member 114 surrounding at least a portion of the coil 102 and at least being partially covered by the guard member 104. In some examples, the retention member 114 can comprise a braided material. In some examples, the retention member 114 can include a woven material. In addition, the retention member 114 can provide a surface area that encourages or promotes tissue ingrowth and/or adherence, and/or reduce trauma to native tissue. For example, in certain instances, the retention member 114 can have a textured outer surface configured to promote tissue ingrowth. In certain instances, the retention member 114 can be impregnated with growth factors to stimulate or promote tissue ingrowth.

In some examples, as illustrated in FIGS. 5A-5B and 7A-7B, at least a proximal end portion of the retention member 114 can extend out of a proximal end of the guard member 104. In some examples, at least a distal end portion of the retention member 114 can extend out of a distal end of the guard member 104. In one example, the retention member 114 can be completely covered by the guard member 104.

In some examples, the retention member 114 can be configured to interact with the guard member 104 to limit or resist motion of the guard member 104 relative to the coil 102. For example, a proximal end 105 of the guard member 104 can have an inner diameter that is about the same as an outer diameter of the retention member 114. As such, an inner surface of the guard member 104 at the proximal end 105 can frictionally interact or engage with the retention member 114 so that axial movement of the proximal end 105 of the guard member 104 relative to the coil 102 can be impeded by a frictional force exerted by the retention member 114.

The coil 102 has a proximal end 102p and a distal end 102d (which also respectively define the proximal and distal ends of the docking device 100). When being disposed within the delivery sheath (for example, during delivery of the docking device into the vasculature of a patient), a body of the coil 102 between the proximal end 102p and distal end 102d can form a generally straight delivery configuration (that is, without any coiled or looped portions, but can be flexed or bent) so as to maintain a small radial profile when moving through a patient's vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present), as described above.

The docking device 100 can be releasably coupled to a delivery apparatus. For example, in certain examples, the docking device 100 can be coupled to a delivery apparatus via a release suture that can be configured to be tied to the docking device 100 and cut for removal. In one example, the release suture can be tied to the docking device 100 through an eyelet or eyehole 103 located adjacent the proximal end 102p of the coil. In another example, the release suture can be tied around a circumferential recess that is located adjacent the proximal end 102p of the coil 102.

In some examples, the docking device 100 in the deployed configuration can be configured to fit at the mitral valve position. In other examples, the docking device can also be shaped and/or adapted for implantation at other native valve positions as well, such as at the tricuspid valve. As described herein, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIGS. 5A-5B, the coil 102 in the deployed configuration can include a leading turn 106 (or “leading coil”), a central region 108, and a stabilization turn 110 (or “stabilization coil”) around the central longitudinal axis 101. The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns, such as a proximal turn 108p in connection with the stabilization turn 110, a distal turn 108d in connection with the leading turn 106, and one or more intermediate turns 108m disposed between the proximal turn 108p and the distal turn 108d. In the example shown in FIG. 5A, there is only one intermediate turn 108m between the proximal turn 108p and the distal turn 108d. In other examples, there are more than one intermediate turns 108m between the proximal turn 108p and the distal turn 108d. Some of the helical turns in the central region 108 can be full turns (that is, rotating 360 degrees). In some examples, the proximal turn 108p and/or the distal turn 108d can be partial turns (for example, rotating less than 360 degrees, such as 180 degrees, 270 degrees, etc.).

A size of the docking device 100 can be generally selected based on the size of the desired prosthetic valve to be implanted into the patient. In certain examples, the central region 108 can be configured to retain a radially expandable prosthetic valve (as shown in FIGS. 7A-7B). For example, the inner diameter of the helical turns in the central region 108 can be configured to be smaller than an outer diameter of the prosthetic valve when the prosthetic valve is radially expanded so that additional radial force can act between the central region 108 and the prosthetic valve to hold the prosthetic valve in place. The helical turns (for example, 108p, 108m, 108d) in the central region 108 can also be referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the native annulus, native valve plane, and/or native chamber for better stabilization. In some examples, the stabilization turn 110 can be a full turn (that is, rotating about 360 degrees). In some examples, the stabilization turn 110 can be a partial turn (for example, rotating between about 180 degrees and about 270 degrees).

In one particularly example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

In some examples, the stabilization turn 110 can have an atrial portion 110a in connection with the proximal turn 108p of the central region 108, a stabilization portion 110c adjacent to the proximal end 102p of the coil 102, and an ascending portion 110b located between the atrial portion 110a and the stabilization portion 110c. Both the atrial portion 110a and the stabilization portion 110c can be generally parallel to the helical turns in the central region 108, whereas the ascending portion 110b can be oriented to be angular relative to the atrial portion 110a and the stabilization portion 110c. For example, in certain examples, the ascending portion 110b and the stabilization portion 110c can form an angle from about 45 degrees to about 90 degrees (inclusive). In certain examples, the stabilization portion 110c can define a plane that is substantially parallel to a plane defined by the atrial portion 110a. A boundary 107 (marked by a dashed line in FIG. 5A) between the ascending portion 110b and the stabilization portion 110c can be determined as a location where the ascending portion 110b intersects the plane defined by the stabilization portion 110c. The curvature of the stabilization turn 110 can be configured so that the atrial portion 110a and the stabilization portion 110c are disposed on approximately opposite sides when the docking device 100 is fully expanded. When implanting the docking device 100 at the native mitral valve location, the atrial portion 110a can be configured to abut the posterior wall of the left atrium and the stabilization portion 110c can be configured to flare out and press against the anterior wall of the left atrium.

As noted above, the leading turn 106 can have a larger radial dimension than the helical turns in the central region 108. As described herein, the leading turn 106 can help more easily guide the coil 102 around and/or through the chordae tendineae and/or adequately around all native leaflets of the native valve (for example, the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (that is, rotating about 360 degrees). In some examples, the leading turn 106 can be a partial turn (for example, rotating between about 180 degrees and about 270 degrees). When a prosthetic valve is radially expanded within the central region 108 of the coil, the functional turns in the central region 108 can be further radially expanded. As a result, the leading turn 106 can be pulled in the proximal direction and become a part of the functional turn in the central region 108.

In certain examples, at least a portion of the coil 102 can be surrounded by a first cover 112. As shown in FIGS. 5C-5D, the first cover 112 can have a tubular shape and thus can also be referred to as a “tubular member.” In certain examples, the tubular member 112 can cover an entire length of the coil 102. In certain examples, the tubular member 112 covers only selected portion(s) of the coil 102.

In certain examples, the tubular member 112 can be coated on and/or bonded on the coil 102. In certain examples, the tubular member 112 can be a cushioned, padded-type layer protecting the coil. The tubular member 112 can be constructed of various native and/or synthetic materials. In one particularly example, the tubular member 112 can include expanded polytetrafluoroethylene (ePTFE). In certain examples, the tubular member 112 is configured to be fixedly attached to the coil 102 (for example, by means of textured surface resistance, suture, glue, thermal bonding, or any other means) so that relative axial movement between the tubular member 112 and the coil 102 is restricted or prohibited.

In some examples, as illustrated in FIGS. 5C-5D, at least a portion of the tubular member 112 is surrounded by the retention member 114. In some examples, the tubular member 112 can extend through an entire length of the retention member 114. In some examples, at least a portion of the tubular member 112 may not be surrounded by the retention member 114.

In some examples, a distal end portion of the retention member 114 can extent axially beyond (that is, positioned distal to) the distal end of the guard member 104, and a proximal end portion of the retention member 114 can extend axially beyond (that is, positioned proximal to) the proximal end 105 of the guard member 104 to aid retention of prosthetic valve and tissue ingrowth. In some examples, a distal end of the retention member 114 can be positioned adjacent the leading turn 106 (for example, near the location marked by the dashed line 109 in FIG. 5A). For example, the retention member 114 can cover the functional turns of the coil 102 in the central region 108. Thus, when the docking device 100 is deployed at the native valve and the prosthetic valve is radially expanded within the docking device 100, the retention member 114 at the central region 108 can frictionally engage the prosthetic valve. In another example, the distal end of the retention member 114 can be disposed at or adjacent the distal end 102d of the coil 102. In the example depicted in FIGS. 5A-5B, a proximal end of the retention member 114 can be disposed at or adjacent the ascending portion 110b of the coil 102.

In certain examples, the docking device 100 can have one or more seating markers. For example, FIGS. 5A-5B show a proximal seating marker 121p and a distal seating marker 121d, wherein the proximal seating marker 121p is positioned proximal relative to the distal seating marker 121d. Both the proximal and distal seating markers 121p, 121d can have predefined locations relative to the coil 102. As shown, both the proximal and distal seating markers 121p, 121d can be disposed distal to the ascending portion 110b, for example, at the atrial portion 110a, of the coil 102.

In certain examples, both the proximal and distal seating markers 121p, 121d can include a radiopaque material so that these seating markers can be visible under fluoroscopy such as during an implantation procedure. The seating markers 121p, 121d can be used to mark the proximal and distal boundaries of a segment of the coil 102 where the proximal end 105 of the guard member 104 can be positioned when deploying the docking device 100.

In certain examples, the seating markers 121p, 121d can be disposed on the tubular member 112 and covered by the retention member 114. In some examples, the seating markers 121p, 121d can be disposed on the atrial portion 110a of the coil 102 and covered by the tubular member 112. In particularly examples, the seating markers 121p, 121d can be disposed directly on the retention member 114. In yet alternative examples, the seating markers 121p, 121d can be disposed on different layers relative to each other. For example, one of the seating markers (for example, 121p) can be disposed outside the tubular member 112 and covered by the retention member 114, whereas another seating marker (for example, 121d) can be disposed directly on the coil 102 and covered by the tubular member 112.

In certain examples, a segment of the coil 102 located between the proximal seating marker 121p and the distal seating marker 121d can have an axial length between about 2 mm and about 7 mm, or between about 3 mm and about 5 mm. In one specific example, the axial length of the coil segment between the proximal seating marker 121p and the distal seating marker 121d is about 4 mm.

In certain examples, an axial distance between the proximal seating marker 121p and a distal end of the ascending portion 110b is between about 10 mm and about 30 mm, or between about 15 mm and about 25 mm. In one specific example, the axial distance between the proximal seating marker 121p and the distal end of the ascending portion 110b is about 20 mm.

Although two seating markers 121p, 121d are shown in FIGS. 5A-5B, it is to be understood that the number of seating markers can be more than two or less than two. For example, in one example, the docking device 100 can have only one seating marker (for example, 121p). In another example, one or more additional seating markers can be placed between the proximal and distal seating markers 121p, 121d. As noted above, the proximal end 105 of the guard member can be positioned between the proximal and distal seating markers 121p, 121d when deploying the docking device 100. As such, these additional seating markers can function as a scale to indicate a precise location of the proximal end 105 of the guard member 104 relative to the coil 102.

As described herein, the guard member 104 can constitute a part of a cover assembly 120 for the docking device 100. In some examples, the cover assembly 120 can also include the tubular member 112. In some examples, the cover assembly 120 can further include the retention member 114.

In some examples, as shown in FIGS. 5A-5B, when the docking device 100 is in the deployed configuration, the guard member 104 can be configured to cover a portion (for example, the atrial portion 110a) of the stabilization turn 110 of the coil 102. In certain examples, the guard member 104 can be configured to cover at least a portion of the central region 108 of the coil 102, such as a portion of the proximal turn 108p. In certain examples, the guard member 104 can extend over the entirety of the coil 102.

As described herein, the guard member 104 can radially expand so as to help preventing and/or reducing paravalvular leakage. Specifically, the guard member 104 can be configured to radially expand such that an improved seal is formed closer to and/or against a prosthetic valve deployed within the docking device 100. In some examples, the guard member 104 can be configured to prevent and/or inhibit leakage at the location where the docking device 100 crosses between leaflets of the native valve (for example, at the commissures of the native leaflets). For example, without the guard member 104, the docking device 100 may push the native leaflets apart at the point of crossing the native leaflets and allow for leakage at that point (for example, along the docking device or to its sides). However, the guard member 104 can be configured to expand to cover and/or fill any opening at that point and inhibit leakage along the docking device 100.

In another example, when the docking device 100 is deployed at a native atrioventricular valve, the guard member 104 covers predominantly a portion of the stabilization turn 110 and/or a portion of the central region 108. In one example, the guard member 104 can cover predominantly the atrial portion 110a of the stabilization turn 110 that is located distal to the ascending portion 110b. Thus, the guard member 104 does not extend into the ascending portion 110b (or at least the guard member 104 can terminate before the anterolateral commissure of the native valve) when the docking device 100 is in the deployed configuration. In certain circumstances, the guard member 104 can extend onto the ascending portion 110b. This may cause the guard member 104 to kink, which (in some instances) may reduce the performance and/or durability of the guard member. Thus, the retention member 114 can, among other things, improve the functionality and/or longevity of the guard member 104 by preventing the guard member 104 from extending into the ascending portion 110b of the coil 102.

Yet in alternative examples, the guard member 104 can cover not only the atrial portion 110a, but can also extend over the ascending portion 110b of the stabilization turn 110. This can occur, for example, in circumstances when the docking device is implanted in other anatomical locations and/or the guard member 104 is reinforced to reduce the risk of wire break.

In various examples, the guard member 104 can help covering an atrial side of an atrioventricular valve to prevent and/or inhibit blood from leaking through the native leaflets, commissures, and/or around an outside of the prosthetic valve by blocking blood in the atrium from flowing in an atrial to ventricular direction (that is, antegrade blood flow)—other than through the prosthetic valve. Positioning the guard member 104 on the atrial side of the valve can additionally or alternatively help reduce blood in the ventricle from flowing in a ventricular to atrial direction (that is, retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (that is, retrograde blood flow). Positioning the guard member 104 on the ventricular side of the valve can additionally or alternatively help reduce blood in the atrium from flowing in the atrial direction to ventricular direction (that is, antegrade blood flow)—other than through the prosthetic valve.

The guard member 104 can include an expandable member 116 and a cover member 118 (also referred to as a “second cover” or an “outer cover”) surrounding an outer surface of the expandable member 116. In certain examples, the expandable member 116 surrounds at least a portion of the tubular member 112. In certain examples, the tubular member 112 can extend (completely or partially) through the expandable member 116.

The expandable member 116 can extend radially outwardly from the coil 102 (and the tubular member 112) and is movable between a radially compressed (and axially elongated) state and a radially expanded (and axially foreshortened) state. That is, the expandable member 116 can axially foreshorten when it moves from the radially compressed state to the radially expanded state and can axially elongate when it moves from the radially expanded state to the radially compressed state.

In certain examples, the expandable member 116 can include a braided structure, such as a braided wire mesh or lattice. In certain examples, the expandable member 116 can include a shape memory material that is shape set and/or pre-configured to expand to a particular shape and/or size when unconstrained (for example, when deployed at a native valve location). For example, the expandable member 116 can have a braided structure containing a shape memory alloy with Superelastic properties, such as Nitinol. In certain examples, the expandable member 116 can have a braided structure containing a ternary shape memory alloy with Superelastic properties, such as NiTiX where X can be chromium (Cr), cobalt (Co), zirconium (Zr), hafnium (Hf), etc. In certain examples, the expandable member 116 can comprise a metallic material that does not have the shape memory properties. Examples of such metallic material include cobalt-chromium, stainless steel, etc. In one specific example, the expandable member 116 can comprise nickel-free austenitic stainless steel in which nickel can be completely replaced by nitrogen. In another specific example, the expandable member 116 can comprise cobalt-chromium or cobalt-nickel-chromium-molybdenum alloy with significantly low density of titanium. The number of wires (or fibers, strands, or the like) forming the braided structure can be selected to achieve a desired elasticity and/or strength of the expandable member 116. In certain examples, the number of wires used to braid the expandable member 116 can range from 16 to 128 (for example, 32 wires, 48 wires, 64 wires, 96 wires, etc.). In certain examples, the braid density can range from 20 picks per inch (PPI) to 70 PPI, or from 25 PPI to 65 PPI. In one specific example, the braid density is about 36 PPI. In another specific example, the braid density is about 40 PPI. In certain examples, the diameter of the wires can range from about 0.002 inch to about 0.004 inch. In one particularly example, the diameter of the wires can be about 0.003 inch. In another example, the expandable member 116 can be a combination of braided wire (which can include a shape memory material or non-shape memory material) and a polymeric material and/or textile (for example, polyethylene terephthalate (PET), polytetrafluoroethylene (PTFE), polyether ether ketone (PEEK), thermoplastic polyurethane (TPU), etc.). For example, the expandable member 116 can include a braided wireframe embedded in a polymeric material.

In some examples, the expandable member 116 can include a braided metallic wireframe coated with an elastomer (for example, ePTFE, TPU, or the like), which can elastically deform as the braided wireframe expands and/or compresses. In some examples, the expandable member 116 can comprise a braid and/or weave that includes one or more metallic wires and one or more polymeric fibers. In other words, the metallic wires and the polymeric fibers can be interwoven together to define a braided structure. In some instances, the polymeric fibers can have the same or about the same diameter as the metallic wires. In other instances, the polymeric fibers can have a smaller diameter (for example, microfibers) than the metallic wires, or vice versa.

In yet another example, the expandable member 116 can include a polymeric material, such as a thermoplastic material (for example, PET, polyether ether ketone (PEEK), thermoplastic polyurethane (TPU), etc.), without a braided wireframe.

In certain examples, the expandable member 116 can include a foam structure. For example, the expandable member can include an expandable memory foam which can expand to a specific shape or specific pre-set shape upon removal of a crimping pressure (for example, removal of the docking device 100 from the delivery sheath) prior to delivery of the docking device.

As described herein, the cover member 118 can be configured to be so elastic that when the expandable member 116 moves from the radially compressed (and axially elongated) state to the radially expanded (and axially foreshortened) state, the cover member 118 can also radially expand and axially foreshorten together with the expandable member 116. In other words, the guard member 104, as a whole, can move from a radially compressed (and axially elongated) state to a radially expanded (and axially foreshortened) state. As described herein, the radially expanded (and axially foreshortened) state is also referred to as the “relaxed state,” and the radially compressed (and axially elongated) state is also referred to as the “collapsed state.”

In certain examples, the cover member 118 can be configured to be atraumatic to native tissue and/or promote tissue ingrowth into the cover member 118. For example, the cover member 118 can have pores to encourage tissue ingrowth. In another example, the cover member 118 can be impregnated with growth factors to stimulate or promote tissue ingrowth, such as transforming growth factor alpha (TGF-alpha), transforming growth factor beta (TGF-beta), basic fibroblast growth factor (bFGF), vascular epithelial growth factor (VEGF), and combinations thereof. The cover member 118 can be constructed of any suitable material, including foam, cloth, fabric, and/or polymer, which is flexible to allow for compression and expansion of the cover member 118. In one example, the cover member 118 can include a fabric layer constructed from a thermoplastic polymer material, such as polyethylene terephthalate (PET).

As described herein, a distal end portion 104d of the guard member 104 (including a distal end portion of the expandable member 116 and a distal end portion of the cover member 118) can be fixedly coupled to the coil 102 (for example, via suturing, gluing, or the like), and a proximal end portion 104p of the guard member 104 (including a proximal end portion of the expandable member 116 and a proximal end portion of the cover member 118) can be axially movable relative to the coil 102. Further, the proximal end portion of the expandable member 116 can be fixedly coupled to the proximal end portion of the cover member 118 (for example, via suturing, gluing, thermal compression, laser fusion, etc.).

Alternatively, the proximal end portion 104p of the guard member 104 can be fixedly coupled to the coil 102, while a distal end portion 104d of the guard member 104 can be axially movable relative to the coil 102.

When the docking device 100 is retained within the delivery sheath in the substantially straight configuration, the expandable member 116 can be radially compressed by the delivery sheath and remains in the radially compressed (and axially elongated) state. The radially compressed (and axially elongated) expandable member 116 can contact the retention member 114 (FIG. 5D) so that no gap or cavity exists between the retention member 114 and the expandable member 116.

After the docking device 100 is removed from the delivery sheath and changes from the delivery configuration to the deployed configuration, the guard member 104 can also move from a delivery configuration to a deployed configuration. In certain examples, a dock sleeve can be configured to cover and retain the docking device 100 within the delivery sheath when navigating the delivery sheath through the patient's native valve. The docking sleeve can also, for example, help to guide the docking device around the native leaflets and chordae. Retraction of the dock sleeve relative to the docking device 100 can expose the guard member 104 and cause it to move from the delivery configuration to the deployed configuration. Specifically, without the constraint of the delivery sheath and the dock sleeve, the expandable member 116 can radially expand (and axially foreshorten) so that a gap or cavity 111 can be created between the retention member 114 and the expandable member 116 (FIG. 5C). Thus, when the guard member 104 is in the delivery configuration, an outer edge of the guard member 104 can extend along and adjacent the coil 102 (since there is no gap 111, only the retention member 114 and/or the tubular member 112 separate the coil 102 from the expandable member 116, as shown in FIG. 5D). When the guard member 104 is in the deployed configuration, the outer edge of the guard member 104 can form a helical shape rotating about the central longitudinal axis 101 (FIGS. 5A-5B and 7A-7B) and at least a segment of the outer edge of guard member can extend radially away from the coil 102 (for example, due to the creation of the gap 111 between the expandable member 116 and the retention member 114).

Because the distal end portion 104d of the guard member 104 is fixedly coupled to the coil 102 and the proximal end portion 104p of the guard member 104 can be axially movable relative to the coil 102, the proximal end portion 104p of the guard member 104 can slide axially over the tubular member 112 and toward the distal end 102d of the coil 102 when expandable member 116 moves from the radially compressed state to the radially expanded state. As a result, the proximal end portion 104p of the guard member 104 can be disposed closer to the proximal end 102p of the coil 102 when the expandable member 116 is in the radially compressed state than in the radially expanded state.

In certain examples, the cover member 118 can be configured to engage with the prosthetic valve deployed within the docking device 100 so as to form a seal and reduce paravalvular leakage between the prosthetic valve and the docking device 100 when the expandable member 116 is in the radially expanded state. The cover member 118 can also be configured to engage with the native tissue (for example, the native annulus and/or native leaflets) to reduce PVL between the docking device and/or the prosthetic valve and the native tissue.

In certain examples, when the expandable member 116 is in the radially expanded state, the proximal end portion 104p of the guard member 104 can have a tapered shape as shown in FIGS. 5A-5B, such that the diameter of the proximal end portion 104p gradually increases from a proximal end 105 of the guard member 104 to a distally located body portion of the guard member 104. This can, for example, help to facilitate loading the docking device into a delivery sheath of the delivery apparatus and/or retrieval and/or re-positioning of the docking device into the delivery apparatus during an implantation procedure. In addition, due to its small diameter, the proximal end 105 of the guard member 104 can frictionally engage with the retention member 114 so that the retention member 114 can reduce or prevent axial movement of the proximal end portion 104p of the guard member 104 relative to the coil 102.

In certain examples, the docking device 100 can include at least one radiopaque marker configured to provide visual indication about the location of the docking device 100 relative to its surrounding anatomy, and/or the amount of radial expansion of the docking device 100 (for example, when a prosthetic valve is subsequently deployed in the docking device 100) under fluoroscopy. For example, one or more radiopaque markers can be placed on the coil 102. In one particularly example, a radiopaque marker (which can be larger than the seating markers 121p, 121d) can be disposed at the central region 108 of the coil. In another example, one or more radiopaque markers can be placed on the tubular member 112, the expandable member 116, and/or the cover member 118. As noted above, the docking device 100 can also have one or more radiopaque markers (for example, 121p and/or 121d) located distal to the ascending portion 110b of the coil 102. The radiopaque marker(s) used to provide visual indication about the location and/or the amount of radial expansion of the docking device 100 can be in addition to the seating markers (for example, 121p, 121d) described above.

Further details of the docking device and its variants, including various examples of the coil, the first cover (or tubular member), the second cover (or cover member), the expandable member, and other components of the docking device, are described in PCT Patent Application Publication No. WO/2020/247907, the entirety of which is incorporated by reference herein.

Example methods of assembling the guard member 104 are described in PCT Patent Application Publication No. WO/2022/087336 and WO/2023/059513, each of which is incorporated by reference herein in its entirety.

### Exemplary Prosthetic Valves

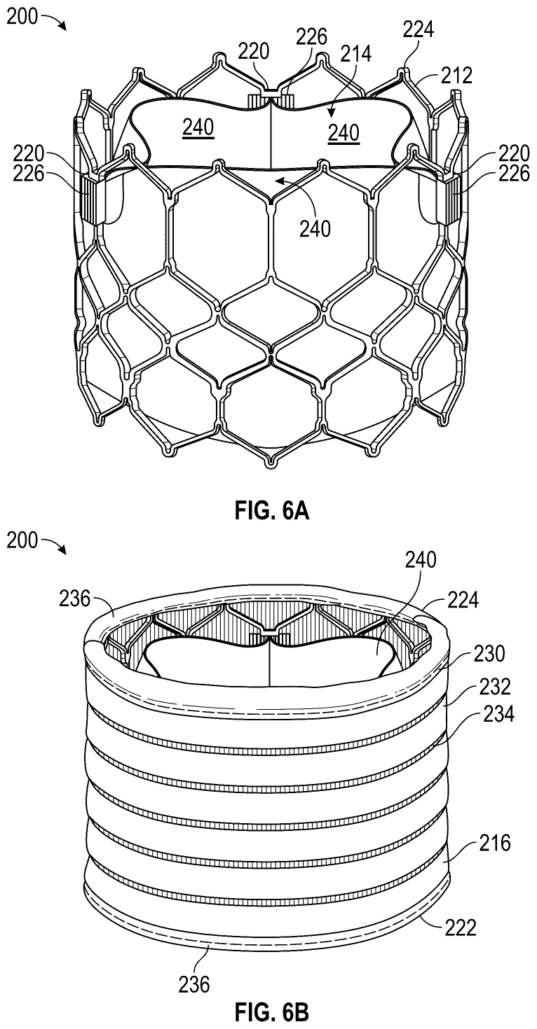

FIGS. 6A-6B show a prosthetic valve 200, according to one example. The prosthetic valve 200 can be adapted to be implanted, with or without a docking device, in a native valve annulus, such as the native mitral valve annulus, native aortic annulus, native pulmonary valve annulus, etc. The prosthetic valve 200 can include a frame 212, a valvular structure 214, and a valve cover 216 (the valve cover 216 is removed in FIG. 6A to show the frame structure).

The valvular structure 214 can include three leaflets 240, collectively forming a leaflet structure (although a greater or fewer number of leaflets can be used), which can be arranged to collapse in a tricuspid arrangement. The leaflets 240 are configured to permit the flow of blood from an inflow end 222 to an outflow end 224 of the prosthetic valve 200 and block the flow of blood from the outflow end 224 to the inflow end 222 of the prosthetic valve 200. The leaflets 240 can be secured to one another at their adjacent sides to form commissures 226 of the leaflet structure. The lower edge of valvular structure 214 desirably has an undulating, curved scalloped shape. By forming the leaflets 240 with this scalloped geometry, stresses on the leaflets 240 can be reduced, which in turn can improve durability of the prosthetic valve 200. Moreover, by virtue of the scalloped shape, folds and ripples at the belly of each leaflet 240 (the central region of each leaflet), which can cause early calcification in those areas, can be eliminated or at least minimized. The scalloped geometry can also reduce the amount of tissue material used to form leaflet structure, thereby allowing a smaller, more even crimped profile at the inflow end of the prosthetic valve 200. The leaflets 240 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 212 can be formed with a plurality of circumferentially spaced slots, or commissure windows 220 (three in the illustrated example) that are adapted to mount the commissures 226 of the valvular structure 214 to the frame. The frame 212 can be made of any of various suitable plastically expandable materials (for example, stainless steel, etc.) or self-expanding materials (for example, Nitinol) as known in the art. When constructed of a plastically expandable material, the frame 212 (and thus the prosthetic valve 200) can be crimped to a radially compressed state on a delivery apparatus and then expanded inside a patient by an inflatable balloon or equivalent expansion mechanism. When constructed of a self-expandable material, the frame 212 (and thus the prosthetic valve 200) can be crimped to a radially compressed state and restrained in the compressed state by insertion into a valve sheath or equivalent mechanism of a delivery apparatus. Once inside the body, the prosthetic valve 200 can be advanced from the delivery sheath, which allows the prosthetic valve 200 to expand to its functional size.

Suitable plastically expandable materials that can be used to form the frame 212 include, without limitation, stainless steel, a nickel-based alloy (for example, a cobalt-chromium or a nickel-cobalt-chromium alloy), polymers, or combinations thereof. In particularly examples, frame 212 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form the frame 212 can provide superior structural results over stainless steel. In particular, when MP35N is used as the frame material, less material is needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

As shown in FIG. 6B, the valve cover 216 can include an outer portion 218 which can cover an entire outer surface of the frame 212. In certain examples, as shown in FIG. 7A, the valve cover 216 can also include an inner portion 228. The inner portion 228 can cover an entire inner surface of the frame 212, or alternatively, cover only selected portions of the inner surface of the frame 212. In the depicted example, the inner portion 228 is formed by folding the valve cover 216 over the outflow end 224 of the frame 212. In certain examples, a protective cover 236 comprising a high abrasion resistant material (for example, ePTFE, etc.) can be placed over the fold of the valve cover 216 at the outflow end 224. In certain examples, similar protective cover 236 can be placed over the inflow end 222 of the frame. The valve cover 216 and the protective cover 236 can be affixed to the frame 212 by a variety of means, such as via sutures 230.

As described herein, the valve cover 216 can be configured to prevent paravalvular leakage between the prosthetic valve 200 and the native valve, to protect the native anatomy, to promote tissue ingrowth, among some other purposes. For mitral valve replacement, due to the general D-shape of the mitral valve and relatively large annulus compared to the aortic valve, the valve cover 216 can act as a seal around the prosthetic valve 200 (for example, when the prosthetic valve 200 is sized to be smaller than the annulus) and allows for smooth coaptation of the native leaflets against the prosthetic valve 200.

In various examples, the valve cover 216 can include a material that can be crimped for transcatheter delivery of the prosthetic valve 200 and is expandable to prevent paravalvular leakage around the prosthetic valve 200. Examples of possible materials include foam, cloth, fabric, one or more synthetic polymers (for example, polyethylene terephthalate (PET), polytetrafluoroethylene (PTFE), expanded polytetrafluoroethylene (ePTFE), etc.), organic tissues (for example, bovine pericardium, porcine pericardium, equine pericardium, etc.), and/or an encapsulated material (for example, an encapsulated hydrogel).

In certain examples, the valve cover 216 can be made of a woven cloth or fabric possessing a plurality of floated yarn sections 232 (for example, protruding or puffing sections, also referred to as “floats” hereinafter). Details of exemplary covered valves with a plurality of floats 232 are further described in U.S. Patent Publication Nos. US2019/0374337, US2019/0192296, and US2019/0046314, the disclosures of which are incorporated herein in their entireties for all purposes. In certain examples, the floated yarn sections 232 are separated by one or more horizontal bands 234. In some examples, the horizontal bands 234 can be constructed via a leno weave, which can improve the strength of the woven structure. In some examples of the woven cloth, vertical fibers (for example, running along the longitudinal axis of the prosthetic valve 200) can include a yarn or other fiber possessing a high level of expansion, such as a texturized weft yarn, while horizontal fibers (for example, running circumferentially around the prosthetic valve 200) in a leno weave can include a low expansion yarn or fiber.

In some examples, the valve cover 216 can include a woven cloth resembling a greige fabric when assembled and under tension (for example, when stretched longitudinally on a compressed valve prior to delivery of a prosthetic valve 200). When the prosthetic valve 200 is deployed and expanded, tension on floats 232 is relaxed allowing expansion of the floats 232. In some examples, the valve cover 216 can be heat set to allow floats 232 to return to an enlarged, or puffed, space-filling form. In some examples, the number and sizes of floats 232 can be optimized to provide a level of expansion to prevent paravalvular leakage across the mitral plane (for example, to have a higher level of expansion thickness) and/or a lower crimp profile (for example, for delivery of the prosthetic valve). Additionally, the horizontal bands 234 can be optimized to allow for attachment of the valve cover 216 to the frame 212 based on the specific size or position of struts or other structural elements on the prosthetic valve 200.

Further details of the prosthetic valve 200 and its components are described, for example, in U.S. Pat. Nos. 9,393,110 and 9,339,384, which are incorporated by reference herein. Additional examples of the valve cover are described in PCT Patent Application Publication No. WO/2020/247907.

As described above and illustrated in FIGS. 7A-7B, the prosthetic valve 200 can be radially expanded and securely anchored within the docking device 100.

In certain examples, the coil 102 of the docking device 100 in the deployed configuration can be movable between a first radially expanded configuration before the prosthetic valve 200 is radially expanded within the coil 102 and a second radially expanded configuration after the prosthetic valve 200 is radially expanded within the coil 102. In the example depicted in FIGS. 7A-7B, the coil 102 is in the second radially expanded configuration since the prosthetic valve 200 is shown in the radially expanded state.

As described herein, at least a portion of the coil 102, such as the central region 108, can have a larger diameter in the second radially expanded configuration than in the first radially expanded configuration (that is, the central region 108 can be further radially expanded by radially expanding the prosthetic valve 200). As the central region 108 increases in diameter when the coil 102 moves from the first radially expanded configuration to the second radially expanded configuration, the functional turns in the central region 108 and the leading turn 106 can rotate circumferentially (for example, in clockwise or counter-clockwise direction when viewed from the stabilization turn 110). Circumferential rotation of the functional turns in the central region 108 and the leading turn 106, which can also be referred to as “clocking,” can slightly unwind the helical coil in the central region 108. Generally, the unwinding can be less a turn, or less than a half turn (that is, 180 degrees). For example, the unwinding can be about 60 degrees and may be up to 90 degrees in certain circumstances. As a result, a distance between the proximal end 102p and the distal end 102d of the coil 102 measured along the central longitudinal axis of the coil 102 can be foreshorten.

In the example depicted in FIGS. 7A-7B, the proximal end 105 of the guard member 104 is shown to be positioned distal to the proximal seating marker 121p. In other examples, after the prosthetic valve 200 is radially expanded within the coil 102, the proximal end 105 of the guard member 104 can be positioned proximal to the proximal seating marker 121p (that is, the proximal seating marker 121p is covered by the guard member 104) but remains distal to the ascending portion 110b.

### Exemplary Retention Members

As described above, the retention member (for example, 114) of the docking device can have a number of desired functions. For example, the textured outer surface of the retention member can be configured to encourage or promote tissue ingrowth. The tissue ingrowth can help reducing or preventing migration of the prosthetic valve and docking device relative to the native tissue. The retention member extending out of a proximal end of the guard member can resist motion of the guard member relative to the coil. Additionally, the retention member extending out of a distal end of the guard member can frictionally engage the prosthetic valve that is radially expanded within the docking device so as to anchor the prosthetic valve more securely. However, in certain circumstances, hemolysis may occur when some red blood cells shear against the rough textured outer surface of the retention braid, especially when there is a paravalvular leakage jet at the mitral valve. As described herein, the retention braid can be configured to mitigate or reduce the likelihood of hemolysis while still maintaining the desired functions noted above.

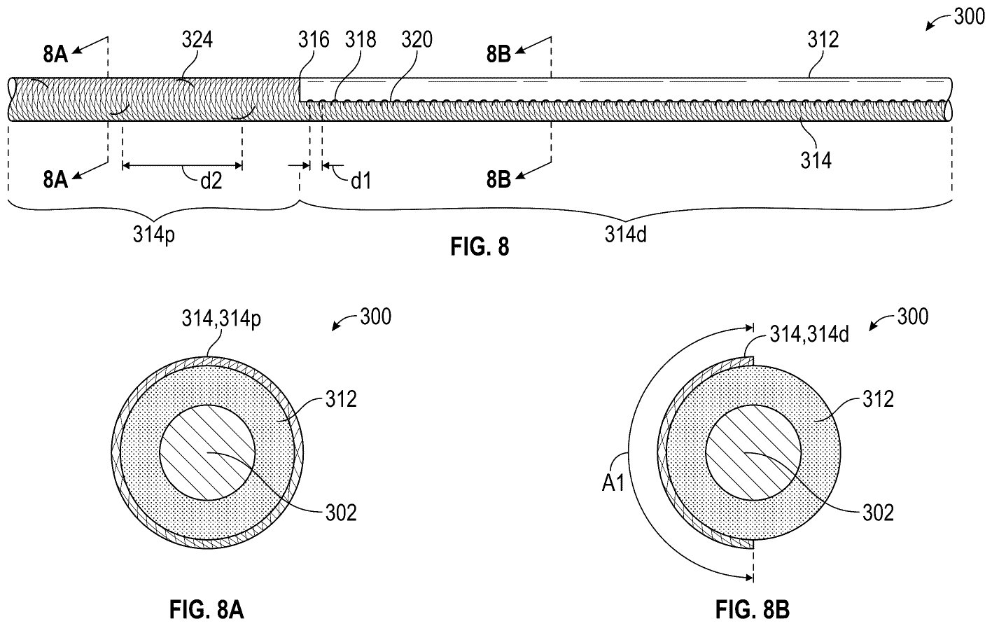

In one example, FIG. 8 depicts a section of a docking device 300 in a substantially straight delivery configuration, and FIGS. 8A-8B show two cross-sections of the docking device 300, respectively. As shown, the docking device 300 includes a coil 302 (similar to 102), a tubular member 312 (similar to 112) covering and fixedly attached to the coil 302, and a retention member 314 covering at least a portion of the tubular member 312 (thus also covering a corresponding portion of the underlying coil 302). As described above, the tubular member 312 can include ePTFE, which provides a biocompatible and relatively smooth surface. In some examples, the retention member 314 has a braided structure, for example, by interweaving a plurality of fibers in a diagonally overlapping pattern. In some examples, the retention member 314 has a woven structure, for example, by interlacing two sets of fibers, the warp and the weft, which are at about the right angle to each other. For clarity, the guard member (similar to 104) of the docking device 300 is removed.

The retention member 314 can have a first axial segment 314d and a second axial segment 314p that are connected at a boundary 316. As shown in FIG. 8A, the second axial segment 314p encloses the full circumference of the coil 302 (and the tubular member 312). Thus, the second axial segment 314p has a generally cylindrical shape. In contrast, as shown in FIG. 8B, the first axial segment 314d encloses only a partial circumference of the coil 302 (and the tubular member 312). Thus, the first axial segment 314d has a longitudinally truncated cylindrical shape (for example, a half shell shape). As a result, a circumferential portion of the coil 302 and the corresponding circumferential portion of the tubular member 312 (extending along the first axial segment 314d) are not covered by the retention member 314.

In some examples, the partial circumference of the coil 302 enclosed by the first axial segment 314d has an arc angle (A1) between 90 degrees and 300 degrees, inclusive. In some examples, A1 is between 120 degrees and 270 degrees, inclusive. In one specific example, A1 is about 180 degrees.

Although not shown, the guard member of the docking device 300 can be attached to the tubular member 312 (and the underlying coil 302) so that the guard member surrounds at least a mid-portion of the retention member 314, similar to the examples depicted in FIGS. 5A-5B and described above.

In certain examples, the second axial segment 314p is located proximal to the first axial segment 314d. For example, when the guard member of the docking device 300 is in the radially expanded state, the first axial segment 314d can extend out of a distal end of the guard member, and the second axial segment 314p can extend out of a proximal end of the guard member. In some examples, a proximal end of the second axial segment 314p can be situated at an ascending portion (similar to 110b) of the coil 302.

In certain examples, the first axial segment 314d is configured to have a predetermined angular position relative to the coil 302 and the tubular member 312. Specifically, when the docking device 300 is deployed at the native valve, at least a portion of the first axial segment 314d is oriented to face radially inwardly so as to frictionally engage the prosthetic valve that is radially expanded within the docking device 300, and a portion of the coil and the corresponding tubular member extending along and not covered by the first axial segment 314d are oriented to face radially outwardly. Because the uncovered portion of the tubular member generally has a smoother surface than the retention member, it will be less likely to damage the red blood cells and thus the risk of hemolysis can be reduced.

The retention member 314 can be prepared in a number of ways. For example, the initial retention member can have a tubular configuration (that is, having a cylindrical shape throughout its longitudinal length). A circumferential portion of the retention member can be removed by first cutting along two longitudinally parallel lines that are circumferentially spaced apart. The two parallel cut lines can extend from a distal end of the tubular retention member to the boundary 316. Then, another circumferential cut across the two parallel cut lines can be made at the boundary 316 to remove the circumferential portion between the cut lines. The removal of the circumferential portion of the retention member can therefore create the longitudinally truncated cylindrical shape of the first axial segment 314d. For attachment, the retention member 314 can slide over the tubular member 312, and then be affixed to the tubular member 312 (and thus the underlying coil 302) via sutures, as described below.

As another example, the initial retention member can similarly have a tubular configuration. A longitudinal cut of the retention member can be made to create a slit extending from a distal end of the tubular retention member to the boundary 316. Then a circumferential cut connected to the slit and with a predefined arc angle can be made at the boundary 316. The cut portion of the retention member can then be folded longitudinally in half. Such folding can therefore create the longitudinally truncated cylindrical shape of the first axial segment 314d. For attachment, the retention member 314 can slide over the tubular member 312, and then be affixed to the tubular member 312 (and the underlying coil 302) via sutures, as described below.

The cutting in any of the examples described above can be laser cutting or other cutting means that sufficiently heat or fuse the fibers of the retention member 314 along the cutting edges so as to create seals to prevent fraying of the fibers.

As another example, the initial retention member can have a flat configuration. For example, both the first axial segment 314d and second axial segment 314p can be configured as rectangular sheets. The width of the second axial segment can be equal to the circumference of the tubular member 312, and the width of the first axial segment is smaller than the circumference of the tubular member 312. For attachment, the sheet of retention member 314 can be wrapped around the tubular member 312 (and the underlying coil 302) so as to create the cylindrical shape of the second axial segment 314p and the longitudinally truncated cylindrical shape of the first axial segment 314d. The retention member 314 can then be affixed to tubular member 312 (and the underlying coil 302) via sutures, as described below.

In certain examples, the first axial segment 314d can be affixed to the coil 302 and the tubular member 312 via a plurality of stitches 318 extending along two longitudinal edges 320 of the first axial segment 314d. For instance, the plurality of stitches 318 can be formed by a continuous in-and-out stitches extending through a thickness of the first axial segment 314d and the underlying tubular member 312. The plurality of stitches 318 can have a very small inter-stitch distance (d1) to prevent fraying of the fibers at the edges 320 and detachment of the retention member 314 from the tubular member 312. In some examples, d1 is between 0.5 mm and 1.5 mm, inclusive. In one specific example, d1 is about 1 mm.

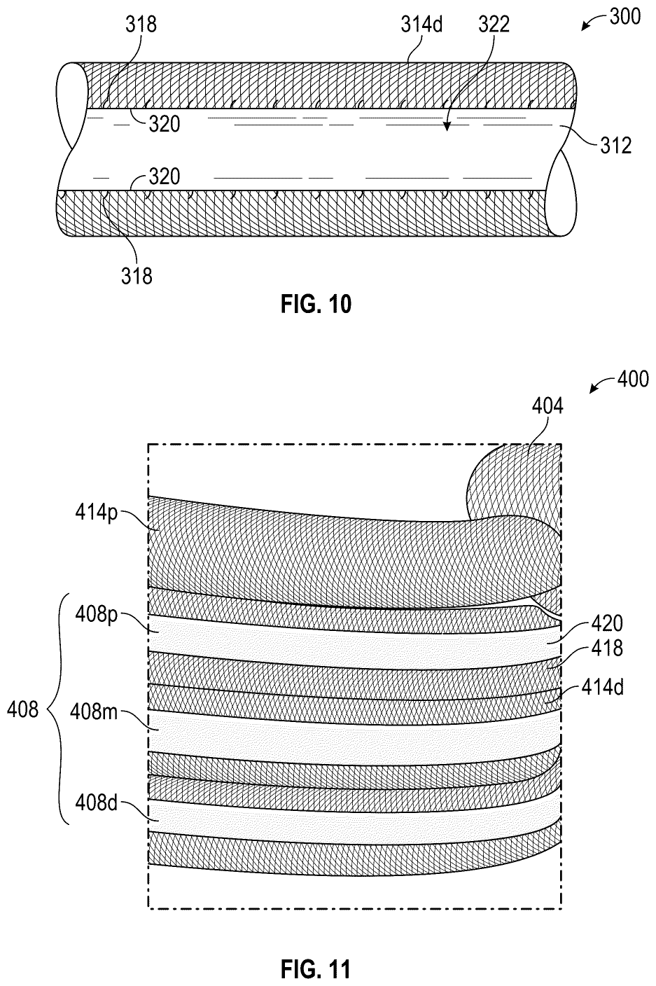

To further illustrate, FIG. 10 is a side view of a portion of the first axial segment 314d when the docking device 300 is in a substantially straight configuration. As shown, the first axial segment 314d has a circumferential gap 322 exposing the underlying tubular member 312. The gap 322 is formed between two longitudinal edges 320 of the first axial segment 314d. In this example, A1 is greater than 180 degrees (thus, the gap 322 has an arc angle that is less than 180 degrees). As shown, the first axial segment 314d is attached to the underlying coil and tubular member by a plurality of stitches 318 extending along the two longitudinal edges 320.

In some examples, the second axial segment 314p can be affixed to the coil 302 via a spiral suture 324 The spiral suture 324 can extend along the entire length of the second axial segment 314p while rotating around a longitudinal axis of the second axial segment 314p. For example, the spiral suture 324 can be formed by a continuous in-and-out stitches extending through a thickness of the second axial segment 314p and the underlying tubular member 312. The spiral suture 324 has a pitch (d2), which is the axial distance for the spiral suture 324 to complete one rotation. In some examples, d2 is between 2 mm and 8 mm, or between 3 mm and 7 mm, or between 4 mm and 6 mm, all inclusive. In one specific example, d2 is about 5 mm.

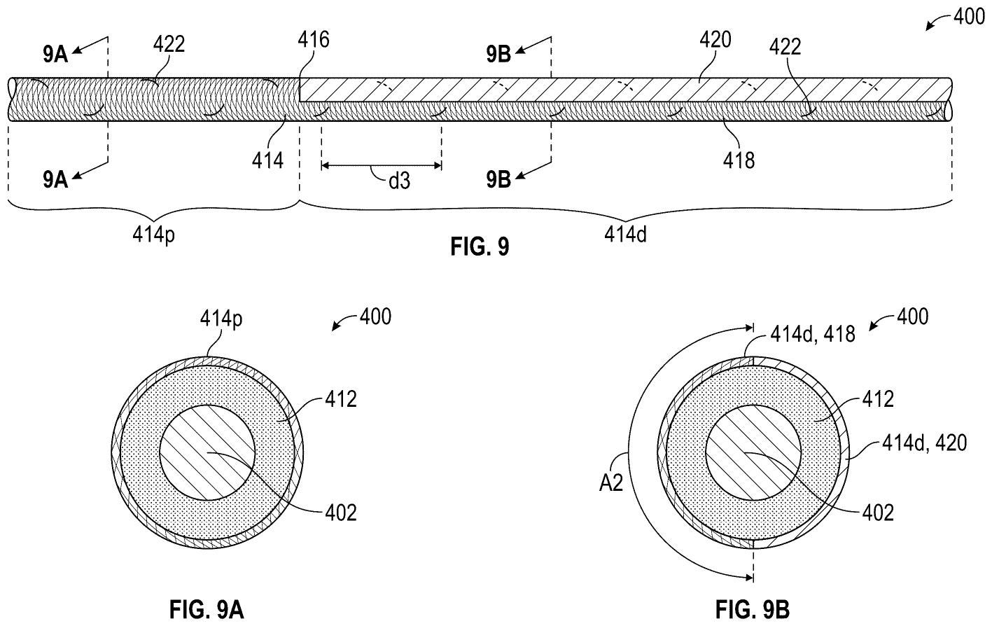

In another example, FIG. 9 depicts a section of a docking device 400 in a substantially straight delivery configuration, and FIGS. 9A-9B show two cross-sections of the docking device 400, respectively. Similarly, the docking device 400 includes a coil 402 (similar to 102), a tubular member 412 (similar to 112) covering and fixedly attached to the coil 402, and a retention member 414 covering at least a portion of the tubular member 412 (thus also covering a corresponding portion of the underlying coil 402). The retention member 414 can comprise a braided material or a woven material. For clarity, the guard member (similar to 104) of the docking device 400 is removed.

The retention member 414 can have a first axial segment 414d and a second axial segment 414p that are connected at a boundary 416. As shown in FIGS. 8A-8B, both the first axial segment 414d and the second axial segment 414p enclose the full circumference of the tubular member 412 (and the underlying coil 402). Thus, the entire retention member 414 has a generally cylindrical shape. In contrast to the second axial segment 414p which has a substantially uniform and textured outer surface in full circumference (360 degrees), the first axial segment 414d has two circumferential portions that exhibit different physical properties. Specifically, the first axial segment 414d includes a first circumferential portion 418 which has a textured (and rough) outer surface similar to the second axial segment 414p, and a second circumferential portion 420 which has a non-textured (and smooth) outer surface. In other words, the second circumferential portion 420 has a smoother outer surface than the first circumferential portion 418 (and the second axial segment 414p).

As described herein, the surface roughness can be measured based on industry standards such as ISO 4287-1:1984, DIN 4762, 4768, etc. For example, the surface roughness can be calculated based on measuring the average or root-mean-square of microscopic peaks and valleys across the surface. In some cases, the surface roughness of an object may be also measured by comparing the surface of the object to a known sample.

In some examples, the first circumferential portion 418 has an arc angle (A2) between 90 degrees and 300 degrees, inclusive. In some examples, A2 is between 120 degrees and 270 degrees, inclusive. In one specific example, A2 is about 180 degrees.

Although not shown, the guard member of the docking device 400 can be attached to the tubular member 412 and the coil 402 so that the guard member surrounds at least a mid-portion of the retention member 414, similar to the examples depicted in FIGS. 5A-5B and described above.

In certain examples, the second axial segment 414p is located proximal to the first axial segment 414d. For example, when the guard member of the docking device 400 is in the radially expanded state, the first axial segment 414d can extend out of a distal end of the guard member, and the second axial segment 414p can extend out of a proximal end of the guard member. In some examples, a proximal end of the second axial segment 414p can be situated at an ascending portion (similar to 110b) of the coil 402.

In certain examples, the first axial segment 414d is configured to have a predetermined angular position relative to the coil 402 and the tubular member 412. Specifically, when the docking device 400 is deployed at the native valve, at least a portion of the first circumferential portion 418 is oriented to face radially inwardly so as to frictionally engage the prosthetic valve that is radially expanded within the docking device 400, and at least a portion of the second circumferential portion 420 is oriented to face radially outwardly. Because the second circumferential portion 420 has a smoother surface than other portions (for example, 418, 414p) of the retention element, it will be less likely to damage the red blood cells and thus the risk of hemolysis can be reduced.

To further illustrate, FIG. 11 is a side view of the docking device 400 in the helical deployed configuration (for example, a plurality of helical turns 408p, 408m, 408d form a central region 408 similar to 108 of FIG. 5A). As shown, the second axial segment 414p extends out of the proximal end of the guard member 404 and the first axial segment 414d extends out of a distal end of the guard member 404. The second axial segment 414p has a textured outer surface around the full circumference. The first axial segment 414d has the first circumferential portion 418 (with a textured outer surface) and the second circumferential portion 420 (with a smoother outer surface). In this example, A2 is greater than 180 degrees (thus, the smooth-surfaced second circumferential portion 420 has an arc angle that is less than 180 degrees). Thus, the second circumferential portion 420 faces radially outwardly and at least a portion of the first circumferential portion 418 (on opposite side of 420) faces radially inwardly. Additionally, the first circumferential portion 418 also covers the top and bottom surfaces of the helical turns (for example, 408p, 408m, 408d). The textured first circumferential portion 418 between the helical turns can provide additional frictional force therebetween to resist their rotational movement relative to one another, for example, when the prosthetic valve is radially expanded within the central region 408. Such additional frictional force between the helical turns can further facilitate retention of the prosthetic valve within the docking device 400.

For attachment, the retention member 414 can slide over the tubular member 412, and then be affixed to the tubular member 412 (and the underlying coil 402) via sutures 422, as depicted in FIG. 9. In some examples, the suture 422 can have a spiral pattern, extending along the entire length of the retention member 414 while rotating around a longitudinal axis of the retention member 414. The spiral suture 422 has a pitch (d3), which can be between 2 mm and 8 mm, or between 3 mm and 7 mm, or between 4 mm and 6 mm, all inclusive. In one specific example, d3 is about 5 mm. In some examples, the spiral suture 422 can have a smaller pitch in the first axial segment 414d than in the second axial segment 414p.

In the second axial segment 414p, the suture 422 can extend through a thickness of the second axial segment 414p and the underlying tubular member 412 (for example, via in-and-out stitches). Thus, the suture 422 is exposed outside the second axial segment 414p, and such exposed suture 422 can contribute to local roughness of the outer surface.

In the first axial segment 414d, the suture 422 still extends through the tubular member 412 so as to stitch together the first axial segment 414d and the tubular member 412. However, the suture 422 can extend through a thickness of the first circumferential portion 418, but not through a thickness of the second circumferential portion 420. In other words, the suture 422 is exposed outside the first circumferential portion 418, but is hidden underneath the second circumferential portion 420. Thus, the exposed suture 422 outside the first circumferential portion 418 can contribute to the local roughness, whereas the second circumferential portion 420 can maintain a smooth outer surface.

The retention member 414 can be prepared in a number of ways. For example, the initial retention member can have a tubular configuration with a braided structure which provides a substantially uniform texture (and roughness) over its outer surface. The smooth-surfaced second circumferential portion 420 can be created by targeted heating to fuse the braided material located at the second circumferential portion 420.

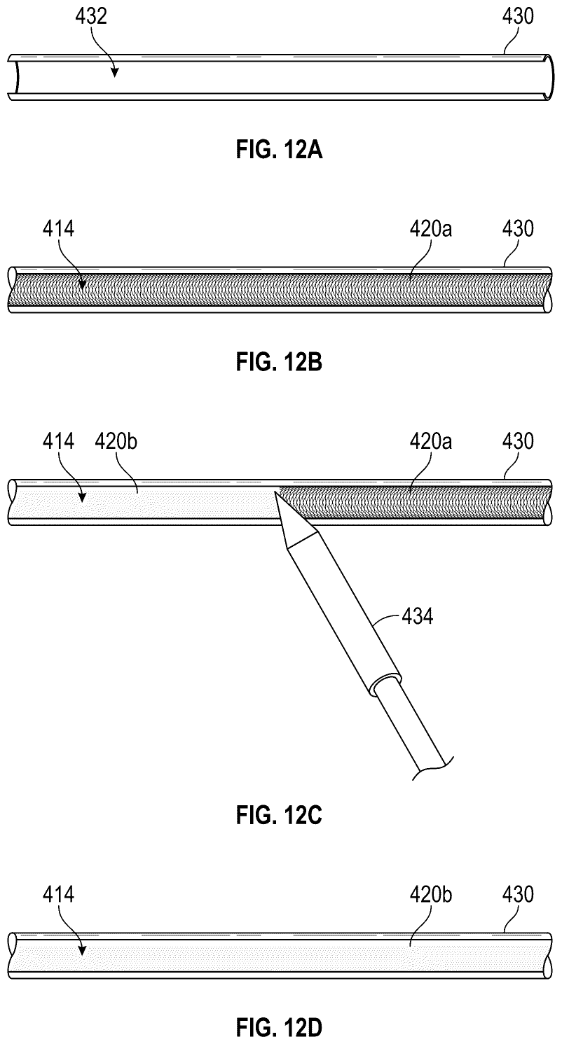

FIGS. 12A-12D illustrate one example method of preparing the retention member 414. Specifically, FIG. 12A depicts an example holder device 430 which has a longitudinally truncated cylindrical shape with a top opening 432. The holder device 430 can comprise a material that has a low thermal conductivity, such as wood, plastics, fiberglass, etc. The inner diameter of the holder device 430 can be configured to match the outer diameter of the retention member 414. FIG. 12B shows placing the retention member 414 in the holder device 430. As described above, the retention member 414 can be attached to and enclose the tubular member 412 and the coil 402 of the docking device 400. At this stage, the retention member 414 has a fully textured outer surface. For example, FIG. 12B shows a circumferential portion 420a of the retention member 414 exposed through the top opening 432 of the holder device 430 has a textured outer surface.

FIG. 12C shows pressing a soldering iron 434 on the exposed circumferential portion 420a of the retention member 414. The soldering iron 434 can be heated to a predetermined temperature and pressed on a target location for a predetermined duration so that the fibers at the target location can be fused together. The soldering iron 434 can move longitudinally along the top opening 432 until the exposed circumferential portion 420a is all heat treated to form a smooth membrane or film 420b, as illustrated in FIG. 12D. Thus, the holder device 430 in this example masks the unexposed circumferential portion of the retention member 414 (that is, the circumferential portion covered by the holder device) from the heat treatment, thereby maintaining its textured (rough) outer surface.

Although the soldering iron 434 is shown in this example as a heating source, it is to be understood that other heating sources (for example, using an ultrasonic welding machine, or other types of heating apparatuses) can be used to fuse the fibers and create the smooth surface portion of the retention member.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking device for securing a prosthetic valve at a native valve, the docking device comprising: a coil comprising a plurality of helical turns when deployed at the native valve; and a retention member covering at least a portion of the coil, wherein the retention member comprises a first axial segment which encloses only a partial circumference of the coil.

Example 2. The docking device of any example herein, particularly example 1, wherein the retention member comprises a second axial segment that encloses a full circumference of the coil.

Example 3. The docking device of any example herein, particularly example 2, wherein the second axial segment is proximal to the first axial segment.

Example 4. The docking device of any example herein, particularly example 3, further comprising an expandable member surrounding at least a portion of the retention member, wherein the expandable member is movable between a radially compressed state and a radially expanded state, wherein when the expandable member is in the radially expanded state, the first axial segment of the retention member extends out of a distal end of the expandable member, and the second axial segment of the retention member extends out of a proximal end of the expandable member.

Example 5. The docking device of any example herein, particularly any one of examples 3-4, wherein a proximal end of the second axial segment is situated at an ascending portion of the coil, wherein when the coil is deployed at the native valve, the ascending portion forms an angle relative to a plane defined by the helical turns, wherein the angle is between 45 degrees and 90 degrees, inclusive.

Example 6. The docking device of any example herein, particularly any one of examples 1-5, wherein retention member comprises a braided material.

Example 7. The docking device of any example herein, particularly any one of examples 1-5, wherein the retention member comprises a woven material.

Example 8. The docking device of any example herein, particularly any one of examples 1-7, wherein the first axial segment is affixed to the coil via a plurality of stitches extending along two longitudinal edges of the first axial segment.

Example 9. The docking device of any example herein, particularly example 8, wherein the plurality of stitches has an inter-stitch distance between 0.5 mm and 1.5 mm, inclusive.

Example 10. The docking device of any example herein, particularly any one of examples 1-9, wherein the partial circumference of the coil enclosed by the first axial segment of the retention member has an arc angle between 90 degrees and 300 degrees, inclusive.

Example 11. The docking device of any example herein, particularly example 10, wherein the arc angle is between 120 degrees and 270 degrees, inclusive.

Example 12. The docking device of any example herein, particularly example 11, wherein the arc angle is 180 degrees.

Example 13. The docking device of any example herein, particularly any one of examples 1-12, wherein when the coil is deployed at the native valve, at least a portion of the first axial segment faces radially inwardly so as to frictionally engage the prosthetic valve that is radially expanded within a central region defined by the helical turns, wherein a portion of the coil extending along and not covered by the first axial segment faces radially outwardly.

Example 14. A docking device for securing a prosthetic valve at a native valve, the docking device comprising: a coil comprising a plurality of helical turns when deployed at the native valve; and a retention member covering at least a portion of the coil, wherein the retention member comprises a first axial segment, the first axial segment comprising a first circumferential portion and a second circumferential portion, wherein the second circumferential portion has a smoother outer surface than the first circumferential portion.

Example 15. The docking device of any example herein, particularly example 14, wherein the retention member comprises a second axial segment enclosing a full circumference of the coil, wherein the second axial segment has at least the same roughness as the first circumferential portion.

Example 16. The docking device of any example herein, particularly example 15, wherein the second axial segment is proximal to the first axial segment.

Example 17. The docking device of any example herein, particularly example 16, further comprising an expandable member surrounding at least a portion of the retention member, wherein the expandable member is movable between a radially compressed state and a radially expanded state, wherein when the expandable member is in the radially expanded state, the first axial segment of the retention member extends out of a distal end of the expandable member, and the second axial segment of the retention member extends out of a proximal end of the expandable member.

Example 18. The docking device of any example herein, particularly any one of examples 16-17, wherein a proximal end of the second axial segment is situated at an ascending portion of the coil, wherein when the coil is deployed at the native valve, the ascending portion forms an angle relative to a plane defined by the helical turns, wherein the angle is between 45 degrees and 90 degrees, inclusive.

Example 19. The docking device of any example herein, particularly any one of examples 14-18, wherein retention member comprises a braided material.

Example 20. The docking device of any example herein, particularly any one of examples 14-18, wherein the retention member comprises a woven material.

Example 21. The docking device of any example herein, particularly any one of examples 14-20, wherein the first axial segment is affixed to the coil via a spiral suture extending along and around a longitudinal axis of the coil, wherein the spiral suture has a pitch between 4 mm and 6 mm, inclusive.

Example 22. The docking device of any example herein, particularly example 21, wherein the spiral suture extends through a thickness of the first circumferential portion, but does not extend through a thickness of the second circumferential portion.

Example 23. The docking device of any example herein, particularly any one of examples 14-22, wherein the first circumferential portion has an arc angle between 90 degrees and 300 degrees, inclusive.

Example 24. The docking device of any example herein, particularly example 23, wherein the arc angle is between 120 degrees and 270 degrees, inclusive.

Example 25. The docking device of any example herein, particularly example 24, wherein the arc angle is 180 degrees.

Example 26. The docking device of any example herein, particularly any one of examples 14-25, wherein when the coil is deployed at the native valve, at least a portion of the first circumferential portion faces radially inwardly so as to frictionally engage the prosthetic valve that is radially expanded within a central region defined by the helical turns, wherein at least a portion of the second circumferential portion faces radially outwardly.

Example 27. A method for assembling a docking device configured to receive a prosthetic valve, the method comprising: preparing a retention member having a first axial segment and a second axial segment; and attaching the retention member to a coil of the docking device so that the first axial segment encloses only a partial circumference of the coil and the second axial segment encloses a full circumference of the coil, wherein the coil is movable from a substantially straight configuration to a helical configuration.

Example 28. The method of any example herein, particularly example 27, wherein the act of preparing the retention member comprises receiving the retention member in a tubular configuration, and removing a circumferential portion of the first axial segment.

Example 29. The method of any example herein, particularly example 27, wherein the act of preparing the retention member comprises receiving the retention member in a tubular configuration, cutting the first axial segment longitudinally, and folding longitudinally the first axial segment in half.

Example 30. The method of any example herein, particularly example 27, wherein the act of preparing the retention member comprises receiving the retention member in a flat configuration, wherein the first axial segment has a smaller width than the second axial segment, wherein the act of attaching comprises wrapping the retention member around the coil.

Example 31. The method of any example herein, particularly any one of examples 27-30, where the act of attaching comprises stitching the retention member to the coil along two longitudinal edges of the first axial segment.

Example 32. The method of any example herein, particularly any one of examples 27-31, wherein the act of attaching comprises orienting the first axial segment relative to the coil such that when the coil is moved to the helical configuration, at least a portion of the first axial segment faces radially inwardly, and a portion of the coil extending along and not covered by the first axial segment faces radially outwardly.

Example 33. The method of any example herein, particularly any one of examples 27-32, further comprising attaching an expandable member to the coil so that the expandable member surrounds at least a mid-portion of the retention member, wherein the expandable member is movable between a radially compressed state and a radially expanded state.

Example 34. A method for assembling a docking device configured to receive a prosthetic valve, the method comprising: preparing a retention member having a tubular configuration, the retention member having a first axial segment and a second axial segment; and attaching the retention member to a coil of the docking device so that the retention member encloses a full circumference of the coil, the coil being movable from a substantially straight configuration to a helical configuration, wherein the first axial segment comprises a first circumferential portion and a second circumferential portion, wherein the second circumferential portion has a smoother outer surface than the first circumferential portion.

Example 35. The method of any example herein, particularly example 34, wherein the retention member comprises a braided material, wherein the act of preparing the retention member comprises heating the second circumferential portion so as to fuse the braided material thereof.

Example 36. The method of any example herein, particularly example 35, wherein the act of heating comprises applying soldering or ultrasonic welding to the second circumferential portion.

Example 37. The method of any example herein, particularly any one of examples 35-36, wherein the act of preparing the retention member comprises masking the first circumferential portion from the heating.

Example 38. The method of any example herein, particularly any one of examples 34-37, wherein the act of attaching comprises affixing the first axial segment to the coil via a spiral suture extending along and around a longitudinal axis of the coil, wherein the spiral suture extends through a thickness of the first circumferential portion, but does not extend through a thickness of the second circumferential portion.

Example 39. The method of any example herein, particularly any one of examples 34-38, wherein the act of attaching comprises orienting the first axial segment relative to the coil such that when the coil is moved to the helical configuration, at least a portion of the first circumferential portion faces radially inwardly, and at least a portion of the second circumferential portion faces radially outwardly.

Example 40. The method of any example herein, particularly any one of examples 34-39, further comprising attaching an expandable member to the coil so that the expandable member surrounds at least a mid-portion of the retention member, wherein the expandable member is movable between a radially compressed state and a radially expanded state.

Example 41. An implant assembly comprising: a radially expandable and compressible prosthetic valve; and a docking device configured to receive the prosthetic valve, wherein the docking device is according to any one of examples 1-26.

Example 42. A method for implanting a prosthetic valve, the method comprising: deploying a docking device at a native valve; and deploying the prosthetic valve within the docking device, wherein the docking device is according to any one of examples 1-26.

Example 43. A method comprising sterilizing the docking device of any example herein, particularly any one of examples 1-26.

Example 44. A method of treating a heart on a simulation, the method comprising: deploying a docking device at a target location; and deploying a prosthetic valve within the docking device; wherein the docking device is according to any one of examples 1-26.

The features described herein with regard to any example can be combined with other features described in any one or more of the other examples, unless otherwise stated. For example, any one or more of the features of one docking device can be combined with any one or more features of another docking device. As another example, any one or more features of one guard member can be combined with any one or more features of another guard member.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

What is claimed is:

1. A docking device for securing a prosthetic valve at a native valve, the docking device comprising:
a coil comprising a plurality of helical turns when deployed at the native valve; and
a retention member covering at least a portion of the coil,
wherein the retention member comprises a first axial segment which encloses only a partial circumference of the coil.

2. The docking device of claim 1, wherein the retention member comprises a second axial segment that encloses a full circumference of the coil.

3. The docking device of claim 2, wherein the second axial segment is proximal to the first axial segment.

4. The docking device of claim 3, further comprising an expandable member surrounding at least a portion of the retention member, wherein the expandable member is movable between a radially compressed state and a radially expanded state, wherein when the expandable member is in the radially expanded state, the first axial segment of the retention member extends out of a distal end of the expandable member, and the second axial segment of the retention member extends out of a proximal end of the expandable member.

5. The docking device of claim 1, wherein retention member comprises a braided material.

6. The docking device of claim 1, wherein the retention member comprises a woven material.

7. The docking device of claim 1, wherein the first axial segment is affixed to the coil via a plurality of stitches extending along two longitudinal edges of the first axial segment.

8. A docking device for securing a prosthetic valve at a native valve, the docking device comprising:
a coil comprising a plurality of helical turns when deployed at the native valve; and
a retention member covering at least a portion of the coil,
wherein the retention member comprises a first axial segment, the first axial segment comprising a first circumferential portion and a second circumferential portion,
wherein the second circumferential portion has a smoother outer surface than the first circumferential portion.

9. The docking device of claim 8, wherein the retention member comprises a second axial segment enclosing a full circumference of the coil, wherein the second axial segment has at least the same roughness as the first circumferential portion.

10. The docking device of claim 9, wherein the second axial segment is proximal to the first axial segment.

11. The docking device of claim 10, further comprising an expandable member surrounding at least a portion of the retention member, wherein the expandable member is movable between a radially compressed state and a radially expanded state, wherein when the expandable member is in the radially expanded state, the first axial segment of the retention member extends out of a distal end of the expandable member, and the second axial segment of the retention member extends out of a proximal end of the expandable member.

12. The docking device of claim 8, wherein the first axial segment is affixed to the coil via a spiral suture extending along and around a longitudinal axis of the coil.

13. The docking device of claim 12, wherein the spiral suture extends through a thickness of the first circumferential portion, but does not extend through a thickness of the second circumferential portion.

14. A method for assembling a docking device configured to receive a prosthetic valve, the method comprising:
preparing a retention member having a first axial segment and a second axial segment; and
attaching the retention member to a coil of the docking device so that the first axial segment encloses only a partial circumference of the coil and the second axial segment encloses a full circumference of the coil, wherein the coil is movable from a substantially straight configuration to a helical configuration.

15. The method of claim 14, wherein the act of preparing the retention member comprises receiving the retention member in a tubular configuration, and removing a circumferential portion of the first axial segment.

16. The method of claim 14, wherein the act of preparing the retention member comprises receiving the retention member in a tubular configuration, cutting the first axial segment longitudinally, and folding longitudinally the first axial segment in half.

17. The method of claim 14, wherein the act of preparing the retention member comprises receiving the retention member in a flat configuration, wherein the first axial segment has a smaller width than the second axial segment, wherein the act of attaching comprises wrapping the retention member around the coil.

18. A method for assembling a docking device configured to receive a prosthetic valve, the method comprising:
preparing a retention member having a tubular configuration, the retention member having a first axial segment and a second axial segment; and
attaching the retention member to a coil of the docking device so that the retention member encloses a full circumference of the coil, the coil being movable from a substantially straight configuration to a helical configuration,
wherein the first axial segment comprises a first circumferential portion and a second circumferential portion, wherein the second circumferential portion has a smoother outer surface than the first circumferential portion.

19. The method of claim 18, wherein the retention member comprises a braided material, wherein the act of preparing the retention member comprises heating the second circumferential portion so as to fuse the braided material thereof.

20. The method of claim 18, wherein the act of attaching comprises affixing the first axial segment to the coil via a spiral suture extending along and around a longitudinal axis of the coil, wherein the spiral suture extends through a thickness of the first circumferential portion, but does not extend through a thickness of the second circumferential portion.
